(19) United States
(12) Patent Application Publication
(10) Pub. No.: US 2011/0166297 A1
(43) Pub. Date: Jul. 7, 2011

(54) MOLECULARLY IMPRINTED SMART POLYMERS (MISPs)

(75) Inventors: Flavio Grynszpan, Mazkeret Batia (IL); Eran Partouche, Petach-Tikva (IL); Vladimir Shkoulev, Rechovot (IL); Alex Aizikovich, Rechovot (IL); Sharon Gazal, Rechovot (IL); Rachel Nakash-Ozeri, Moshav Gimzo-Doar-Na Merkaz (IL); Isaac Zigelboim, Rechovot (IL)

(73) Assignee: Sailo Nanotechnologies Ltd., Nes Ziona (IL)

(21) Appl. No.: 13/062,956

(22) PCT Filed: Sep. 9, 2009
(86) PCT No.: PCT/IL2009/000878

§ 371 Date: Mar. 9, 2011

Related U.S. Application Data
(60) Provisional application No. 61/136,487, filed on Sep. 9, 2008.

(51) Int. Cl. C08F 8/30; C08F 2/06; C08F 4/04; C08F 220/34; C08F 220/18; C08F 222/38; C08F 226/06; C08F 8/00; C07D 498/04; C07D 491/107; C07D 498/10; C07D 493/10

(52) U.S. Cl. 525/326.9; 526/204; 526/212; 525/328.4; 525/330.5; 525/326.7; 525/329.4; 525/326.8; 548/217; 548/410; 548/409; 544/71; 549/265

(57) ABSTRACT

Molecularly imprinted smart polymers (MISPs) are provided herein, as well as novel monomers for preparing MISPs, and processes for preparing MISPs. The MISPs can be used applications such as, for example, detecting/absorbing or isolating biological and non-biological agents. The MISPs described herein comprise responsive monomeric units which undergo a physico-chemical change (e.g., a bond formation or cleavage) in response to an external change, such that the MISP selectively binds to a target molecule and releases a bound target molecule in response to the external change.

### FIELD AND BACKGROUND OF THE INVENTION

The present invention, in some embodiments thereof, relates to novel Molecularly imprinted smart polymers (MISPs) and, more particularly, but not exclusively, to MISPs that can be used in the field of sensing, solid phase separations (e.g., chromatography) and smart polymers, in applications such as, for example, detecting/absorbing or isolating biological and non-biological agents.

Molecularly Imprinted Polymers (MIPs) are polymers which have imprinted cavities that recognize target molecules present during their synthesis. They are produced by using monomers having complementary functionalities to a desired target molecule, which are then polymerized in the presence of that target. Subsequent removal of the target molecules leaves behind binding sites that are both sterically and functionally (i.e., with respect to charge, polarity, hydrogen bonding, etc.) complementary to the target molecule (see, FIG. 1). A high proportion of cross-linker (e.g., 80-90%) is typically used in MIP syntheses to ensure rigidity of the matrix and binding site integrity. As a result of the presence of binding sites which complement the target, the polymers are capable of selectively binding the target. For sensing applications these selectively-binding polymers can then be incorporated onto sensor surfaces as the key recognition element for the original target molecule. When the target molecule comes into contact with the polymer on the sensor surface, the imprinted complementary binding sites match up with the corresponding features on the target molecule, resulting in capture, of the target molecule and, in appropriately designed systems, signal detection. Compared with current antibody-based technology, MIPs are more robust, more reliable, more versatile, and are simple to use, producible in large scales, compatible with nano and microfabrication, and do not require special handling and storage conditions.

Recent developments in MIPs technologies are summarized, for example, in Alexander et al. [J. Mol. Recognit. 2006; 19: 106-180] and Spivak [Drug Delivery Reviews, 2005; 57: 1779-1794].

Sellergren et al. [Chem. Mater., 1998; 10: 4037-4046] describes selective absorption of cholesterol and other steroidal compounds by MIPs.

International Patent Application No. PCT/IL2006/001318 (Publication No. WO/2007/057891) describes selective binding of an ergosterol conjugate by MIPs, allowing the determination of the presence of ergosterol-containing organisms (e.g., fungi).

Many materials have the ability to respond to a given external stimulus and in some cases this behavior can be harnessed for performing a useful task. Such materials are referred to as “smart” if this response is both controlled and timely. Stimuli may include light or other incident radiation, change of temperature, addition or generation of chemical reagents, change in pH, electric current, charges, and others. Responses to the given stimuli include rearrangement of molecular structures, creation of local charges, absorption or emission of photons, and chemical reactions, to mention just a few. Smart materials are generally reversible in nature with the stimulus and the response being interchangeable.

When a MIP is produced from a material which is responsive to light, the binding capabilities of the MIP may be affected by irradiation. Imprinted membranes which are responsive to light are described in Marx-Tibbon and Willner [J. Chem. Soc., Chem. Commun., 1994, 1261-1262]. Responsive MIPs are described by Gomy and Schmitzer [Organic Letters, 2007, 9: 3865-3868]; Gong et al. [Funct. Mater. 2006, 16: 1759-1767]; Takeuchi et al. [Org. Biomol. Chem., 2007, 5: 2368-2374]; Minoura et al. [Macromolecules, 2004, 37: 9571-9576]; and Minoura et al. [Chem. Mater. 2003, 15: 4703-4704].

U.S. Patent Application No. 20090076437 describes an electroactive MIP having a plurality of binding sites capable of binding an imprint molecule, and an electric potential producing member (EPM) capable of producing an electric potential between the EPM and the MIP, whereby when a sufficient potential is produced between the EPM and the MIP, the imprint molecule is released from the binding site.

### SUMMARY OF THE INVENTION

According to an aspect of some embodiments of the present invention, there is provided a molecularly imprinted polymer comprising a plurality of monomeric units, at least one of the monomeric units is a responsive monomeric unit being capable of undergoing a physico-chemical change in response to an external change, the responsive monomeric unit being incorporated within or attached to the molecularly imprinted polymer, the molecularly imprinted polymer being capable of selectively binding to a target molecule and releasing a bound target molecule in response to the external change.

According to an aspect of some embodiments of the present invention, there is provided a process for producing a molecularly imprinted polymer described herein, the process comprising polymerizing a plurality of monomers in the presence of a template molecule, wherein at least one of the monomers is a responsive monomer being capable of undergoing a physico-chemical change in response to the external change, thereby producing the molecularly imprinted polymer, wherein the template molecule is similar or identical to the target molecule.

According to an aspect of some embodiments of the present invention, there is provided a molecularly imprinted polymer produced according to a process described herein.

According to an aspect of some embodiments of the present invention, there is provided a responsive monomer for preparing a molecularly imprinted polymer capable of selectively binding to a target molecule and releasing a bound target molecule in response to an external change, the monomer comprising at least one polymerizable moiety, and a responsive moiety comprising a heteroalicyclic ring, the heteroalicyclic ring comprising a responsive bond linking a carbon atom and a heteroatom, wherein the responsive bond becomes cleaved in response to the external change, such that the external change causes opening of the heteroalicyclic ring.

According to an aspect of some embodiments of the present invention, there is provided a responsive monomer for preparing a molecularly imprinted polymer capable of selectively binding to a target molecule and releasing a bound target molecule in response to an external change, the monomer comprising at least one polymerizable moiety, and a responsive moiety having the general formula III:

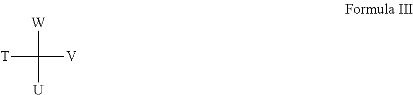

wherein:

T, U and V are each independently selected from the group consisting of substituted or non-substituted aromatic moiety and substituted or non-substituted heteroaromatic moiety; and

W is selected from the group consisting of hydroxy, thiohydroxy, halide, carboxy, and sulfonate.

According to an aspect of some embodiments of the present invention, there is provided a use of a molecularly imprinted polymer described herein for reversibly binding a target molecule of the molecularly imprinted polymer.

According to an aspect of some embodiments of the present invention, there is provided a use of a molecularly imprinted polymer described herein in the manufacture of a product for reversibly binding a target molecule of the molecularly imprinted polymer.

According to an aspect of some embodiments of the present invention, there is provided a method of selectively and reversibly binding a target molecule, the method comprising contacting the molecularly imprinted polymer described herein with the target molecule.

According to some embodiments, the physico-chemical change is selected from the group consisting of formation of a covalent bond linking two atoms in the responsive monomeric unit and cleavage of a covalent bond linking two atoms in the responsive monomeric unit.

According to some embodiments, the external change is selected from the group consisting of a presence and/or change in concentration of a chemical, and a change in pH, an exposure to light, an exposure to irradiation, a temperature change, an exposure to an electric current, and an exposure to an electromagnetic field.

According to some embodiments, the external change is selected from the group consisting of a presence and/or change in concentration of a chemical and a change in pH.

According to some embodiments, the physico-chemical change is reversible.

According to some embodiments, the binding of the molecularly imprinted polymer to the target molecule is via an interaction selected from the group consisting of a covalent bond, an electrostatic bond, and a hydrophobic interaction.

According to some embodiments, the responsive monomeric unit comprises at least one polymerizable moiety and a responsive moiety, the responsive moiety being selected capable of undergoing a physico-chemical change in response to the external change, and the polymerizable moiety forming a polymeric backbone of the polymer by linking the monomeric units in the plurality of monomeric units to one another.

According to some embodiments, the responsive monomer comprises at least one polymerizable moiety and a responsive moiety, the responsive moiety being selected capable of undergoing a physico-chemical change in response to the external change, and the polymerizable moiety being selected capable of linking to other monomers so as to form the polymer.

According to some embodiments, the polymerizing is performed in a mixture of the plurality of monomers and the template molecule.

According to some embodiments, at least some monomers of the plurality of monomers are selected so as to have an affinity to the template molecule and/or the target molecule.

According to some embodiments, the responsive monomer comprises a moiety derived from the target molecule and/or template molecule, wherein after the polymerizing the moiety derived from the target molecule and/or template molecule is released.

According to some embodiments, the responsive moiety comprises a heteroalicyclic ring, the heteroalicyclic ring comprising a responsive bond linking a carbon atom and a heteroatom, wherein the responsive bond cleaves in response to the external change, such that the external change causes opening of the heteroalicyclic ring.

According to some embodiments, the heteroatom of the abovementioned heteroalicyclic ring is selected from the group consisting of N, O and S.

According to some embodiments, the carbon atom which is linked by the responsive bond is further linked to an electron donating moiety.

According to some embodiments, the electron donating moiety comprises at least one electron donating group selected from the group consisting of an amine group, a hydroxy, an alkoxy, a thioalkoxy, a thiohydroxy, an aryloxy, a thioaryloxy and a conjugated pi-electron system.

According to some embodiments, the responsive moiety has the general formula I:

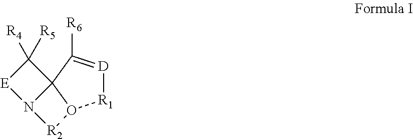

wherein:

the dashed lines denote that the oxygen atom is bound to either R₁ or R₂;

D is selected from the group consisting of N and CR₃;

E is an aromatic or heteroaromatic moiety, being substituted or non-substituted;

R₁ and R₂ are each independently selected from the group consisting of hydrogen, alkyl, alkenyl, alkynyl, cycloalkyl, heteroalicyclic, aryl, arylalkyl, and heteroaryl, or R₁ attaches to R₆ to form a 5- or 6-membered cycloalkyl, heteroalicyclic, aromatic or heteroaromatic ring; and

R₃-R₆ are each independently selected from the group consisting of hydrogen, alkyl, alkenyl, alkynyl, cycloalkyl, heteroalicyclic, aryl, heteroaryl, arylalkyl, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl, or R₃ attaches to R₆ to form a 5- or 6-membered cycloalkyl, heteroalicyclic, aromatic or heteroaromatic ring, with the proviso that neither R₄ nor R₅ is hydrogen.

According to some embodiments, E is phenylene.

According to some embodiments, R₄ and R₅ are each C₁₋₄ alkyl.

According to some embodiments, R₄ and R₅ are each methyl.

According to some embodiments, R₆ is hydrogen.

According to some embodiments, R₁ is selected from the group consisting of a substituted or non-substituted aryl and a substituted or non-substituted heteroaryl, and is being attached to the oxygen atom.

According to some embodiments, D is ═CH—.

According to some embodiments, D is nitrogen.

According to some embodiments, R₁ is nitrophenylene.

According to some embodiments, R₁ is naphthylene.

According to some embodiments, the naphthylene is linked to the polymerizable moiety.

According to some embodiments, R₁ is selected from the group consisting of aryl and heteroaryl.

According to some embodiments, R₁ is nitrophenyl.

According to some embodiments, R₂ is a C₁₋₄ alkyl.

According to some embodiments, R₂ is linked to the polymerizable moiety.

According to some embodiments, R₂ is alkyl, and is attached to the oxygen atom.

According to some embodiments, R₂ is —CH₂CH₂—.

According to some embodiments, E is a benzene ring that is attached to the polymerizable moiety.

According to some embodiments, the responsive moiety has the general formula II:

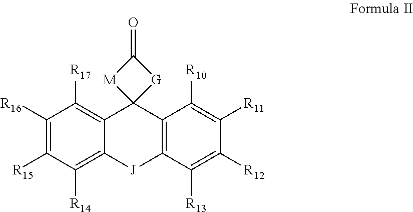

wherein:

G is selected from the group consisting of O, S and NR₁₉;

J is selected from the group consisting of O, S and NR₁₈;

M is an aromatic or heteroaromatic moiety, being substituted or non-substituted; and

R₁₀-R₁₇ are each independently selected from the group consisting of hydrogen, alkyl, alkenyl, alkynyl, cycloalkyl, heteroalicyclic, aryl, heteroaryl, arylalkyl, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl; and

R₁₈ and R₁₉ are each independently selected from the group consisting of hydrogen, alkyl, alkenyl, alkynyl, cycloalkyl, heteroalicyclic, aryl and heteroaryl.

According to some embodiments, M is phenylene.

According to some embodiments, at least one of R₁₂ and R₁₅ is selected from the group consisting of hydroxy, thiohydroxy and amine.

According to some embodiments, R₁₀, R₁₁, R₁₃, R₁₄, R₁₆ and R₁₇ are each hydrogen.

According to some embodiments, J is oxygen.

According to some embodiments, G is NR₁₉ and R₁₉ is alkyl.

According to some embodiments, R₁₉ is alkyl linked to at least one polymerizable moiety.

According to some embodiments, R₁₂ and R₁₅ are each amine.

According to some embodiments, R₁₂ and R₁₅ are each dialkylamine.

According to some embodiments, G is oxygen.

According to some embodiments, R₁₂ and R₁₅ are each hydroxy.

According to some embodiments, M is a benzene ring that is attached to a polymerizable moiety.

According to some embodiments, R₁₂ is hydroxy and R₁₅ is linked to a polymerizable moiety.

According to some embodiments, R₁₅ is selected from the group consisting of alkoxy and aryloxy.

According to some embodiments, R₁₅ is —O—(C₆F₄)—C(═O)—.

According to some embodiments, the responsive moiety has the general formula III:

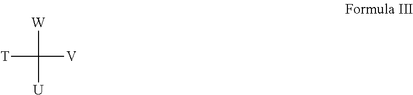

wherein:

T, U and V are each independently selected from the group consisting of substituted or non-substituted aromatic moiety and substituted or non-substituted heteroaromatic moiety; and

W is selected from the group consisting of hydroxy, thiohydroxy, halide, carboxy, and sulfonate.

According to some embodiments, W is hydroxy.

According to some embodiments, the responsive moiety is selected from the group consisting of malachite green, bromocresol green, bromocresol purple and bromothymol blue.

According to some embodiments, the polymerizable moiety of the responsive monomer is selected from the group consisting of vinyl, vinylphenyl, 4-vinylbenzoate, itaconate, acrylate, methacrylate, trifluoromethacrylate, acrylamide and methacrylamide.

According to some embodiments, the responsive monomer is selected from the group consisting of:

A-L-B and

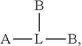

wherein:

A is a responsive moiety;

B is a polymerizable moiety; and

L is absent or is a linker moiety.

According to some embodiments, the responsive monomer comprises a moiety derived from the target molecule, wherein after the polymerizing the moiety derived from the target molecule is released.

According to some embodiments, the moiety derived from the target molecule is attached via a linking group selected from the group consisting of ester, ketal, imine, boronic ester, silyl ether, —O—C(═O)—O— and —O—C(═O)—NH— groups.

According to some embodiments, the responsive monomer is selected from the group consisting of:

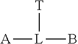

### and T-L-A-L-B

wherein:

A is a responsive moiety;

B is a polymerizable moiety;

L is absent or is a linker moiety; and

T is a moiety derived from the target molecule.

According to some embodiments, the linker is selected from the group consisting of a substituted alkyl, a substituted cycloalkyl, a substituted aryl and a substituted heteroaryl.

According to some embodiments, the abovementioned alkyl, cycloalkyl, aryl, and heteroaryl of the linker are each independently substituted by at least one substituent selected from the group consisting of alkoxy, aryloxy, amine, thioalkoxy, thioaryloxy, amide, carbonyl, carboxy, thiocarboxy, thiocarbonyl, sulfonate, sulfate, urea, disulfide, sulfonyl, sulfinyl, sulfonamide, hydrazine, carbamyl, thiocarbamyl and carbonate.

According to some embodiments, the linker is a substituted aryl, substituted by at least two substituents selected from the group consisting of amine, alkoxy, aryloxy, amide, carbamyl and carbonate.

According to some embodiments, the plurality of monomers comprises, in addition to the responsive monomer, a plurality of monomers comprising at least one polymerizable moiety.

According to some embodiments, the monomers comprising at least one polymerizable moiety each comprise at least one polymerizable moiety selected from the group consisting of vinyl, vinylphenyl, 4-vinylbenzoate, itaconate, 1-vinylimidazole, 2-vinylpyridine, 4-vinylpyridine, 4-vinylimidazole, 4-vinylbenzyl-iminoacetate, acrylate, methacrylate, trifluoromethacrylate, acrylamide and methacrylamide.

According to some embodiments, the monomers comprising at least one polymerizable moiety each comprise at least two polymerizable moieties.

According to some embodiments, the monomers comprising at least two polymerizable moieties are selected from the group consisting of N,N′-methylene-bisacrylamide, divinylbenzene, N,N′-phenylene-bisacrylamide, 2,6-bisacrylamidopyridine, bisphenol A dimethacrylate, trimethylolpropane trimethacrylate, ethylene dimethacrylate (EDMA) and N,O-bismethacryloyl ethanolamine (NOBE).

According to some embodiments, the plurality of monomeric units comprises, in addition to at least one responsive monomeric unit, a plurality of monomeric units which comprise at least one polymerizable moiety for forming a polymeric backbone of the polymer by linking the monomeric units in the plurality of monomeric units to one another.

According to some embodiments, the plurality of monomeric units forms a polymeric backbone selected from the group consisting of poly(styrene), poly(4-vinylbenzoate), poly(itaconate), poly(1-vinylimidazole), poly(2-vinylpyridine), poly(4-vinylpyridine), poly(4-vinylimidazole), poly(4-vinylbenzyl-iminoacetate), poly(acrylate), poly(methacrylate), poly(trifluoromethacrylate), poly(acrylamide), poly(methacrylamide) and copolymers thereof.

According to some embodiments, at least a portion of the monomeric units in the plurality of monomeric units are monomeric units which comprise at least two polymerizable moieties.

According to some embodiments, the monomeric units which comprise at least two polymerizable moieties are selected from the group consisting of N,N′-methylene-bisacrylamide, divinylbenzene, N,N′-phenylene-bisacrylamide, 2,6-bisacrylamidopyridine, bisphenol A dimethacrylate, trimethylolpropane trimethacrylate, ethylene dimethacrylate (EDMA) and N,O-bismethacryloyl ethanolamine (NOBE).

According to some embodiments, the molecularly imprinted polymer is for reversibly binding a target molecule of the molecularly imprinted polymer.

According to some embodiments, the target molecule is a biological or non-biological marker.

According to some embodiments, the target molecule is selected from the group consisting of ergosterol and an adduct of ergosterol.

According to some embodiments, the target molecule is selected from the group consisting of a peptide, a polypeptide, an amino acid, a drug, a hormone, a co-enzyme, a pesticide, an explosive, a carbohydrate, a nucleotide, a polynucleotide, a steroid, and a chemical reagent.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a Background art scheme depicting the preparation of a molecularly imprinted polymer (MIP);

FIG. 2 is a scheme depicting binding and release of a target molecule by a molecularly imprinted smart polymer (MISP), according to some embodiments of the invention;

FIGS. 3A-3C are graphs showing the uptake (in weight percents) over time of an ergosterol-triazolinedione-pyrene adduct (Erg-TAD-Py) by a MIP (MIP-1) and a NIP (non-imprinted polymer) (NIP-1) comprising ergosterol methacrylate functional monomers (FIG. 3A), by a MIP (MIP-2) and a NIP (NIP-2) comprising ergosterol methacrylate and 2-(2,4-dinitrophenylamino)ethyl methacrylate (DNP) functional monomers (FIG. 3B), and by a MIP (MIP-3) and a NIP (NIP-3) comprising DNP functional monomers (FIG. 3C); control polymer (PEDMA) lacked functional monomers;

FIG. 4 is a graph showing the partition coefficient of MIPs comprising ergosterol methacrylate (MIP-1), ergosterol methacrylate and DNP (MIP-2) and DNP (MIP-3) functional monomers for binding of Erg-TAD-Py adduct, ergosterol and ethyl 4-(pyren-1-yl)butanoate (pyrenyl ester);

FIG. 5 presents an image of samples of both forms of exemplary smart polymers comprising a benzospiropyran derivative, a rhodamine B derivative and a fluorescein derivative, as well as the conditions for converting each form to the other;

FIGS. 6A and 6B are graphs showing the uptake of Erg-TAD-Py by exemplary molecularly imprinted smart polymers (MISPs) and non-imprinted smart polymers (NISPs) based on rhodamine B derivatives (FIG. 6A) before (MISP-1, NISP-1) and after (MISP-1(+), NISP-1(+)) activation by acid, and by exemplary MISPs and NISPs based on benzospiropyran derivatives (FIG. 6B) before (MISP-2, NISP-2) and after (MISP-2(UV), NISP-2(UV)) activation by UV light (values for MISP-1 and MISP-2 are defined as 100%);

FIGS. 7A and 7B are graphs showing the uptake (FIG. 7A) and specific uptake (FIG. 7B) of Erg-TAD-Py by an exemplary MISP and NISP based on benzospiropyran derivatives, as well as the specific uptake by the MISP, before (B) and after (A) activation over the course of 2 cycles of activation and deactivation (specific uptake was calculated as the difference between uptake by the MISP and NISP);

FIG. 8 is a graph showing the percentage of bound Erg-TAD-Py released by a non-activated (MISP-2) and activated (MISP-2 (UV)) exemplary benzospiropyran-based MISP;

FIGS. 9A and 9B are graphs showing the uptake of Erg-TAD-Py by exemplary MIPs and NIPs comprising ethylene dimethacrylate (EDMA) and methacrylic acid (MAA) (MIP-2 and NIP-2, FIG. 9A), N,O-bismethacryloyl ethanolamine (NOBE) and methyl methacrylate (MMA) (MIP-1 and NIP-1, FIG. 9A), EDMA without MAA (MIP-2 and NIP-2, FIG. 9B) and NOBE without MMA (MIP-1 and NIP-1, FIG. 9B), over the course of two cycles of treatment with a base (NaOH) and acid (TFA);

FIG. 10 is a graph showing the uptake of Erg-TAD-Py by exemplary MIPs and NIPs comprising EDMA (MIP-2, NIP-2) or NOBE (MIP-1, NIP-1) with bisphenol A dimethacrylate, over the course of two cycles of treatment with a base (NaOH) and acid (TFA);

FIG. 11 is a graph showing uptake of Erg-TAD-Py by an exemplary MIP and NIP, as well as the specific uptake by the MIP, from solutions of 0.05 and 0.01 mg/ml Erg-TAD-Py in ethanol (specific uptake was calculated as the difference between uptake by the MIP and NIP);

FIG. 12 is a graph showing uptake of Erg-TAD-Py by an exemplary MIP and NIP, as well as the specific uptake by the MIP, from solutions of 0.01 mg/ml Erg-TAD-Py in serum or ethanol (EtOH) (specific uptake was calculated as the difference between uptake by the MIP and NIP);

FIG. 13 is a graph showing uptake of Erg-TAD-Py by MIPs and NIPs with (MISP-1, NISP-1) and without (MIP-3, NIP-3) an exemplary indolenine-derived smart monomer, before and after exposure to trifluoroacetic acid; and

FIG. 14 is a graph showing uptake of 9-fluorenyl methanol by a polymer MISP) imprinted using an exemplary “third generation” smart monomer, and by a corresponding non-imprinted polymer (NISP).

### DESCRIPTION OF SPECIFIC EMBODIMENTS OF THE INVENTION

The present invention, in some embodiments thereof, relates to novel Molecularly Imprinted Smart Polymers (MISPs) and, more particularly, but not exclusively, to MISPs that can be used in the field of sensing, solid phase separations (e.g., chromatography) and smart polymers, in applications such as, for example, detecting, absorbing or isolating biological and non-biological agents.

Molecularly imprinted polymers (MIPs) are well-studied in the art, and are used in a variety of applications involving selective binding of a target molecule. The present inventors have envisioned that widespread, practical and convenient use of MIPs is severely limited by the difficulty in separating a bound target from a MIP, which prevents an efficient usage the MIP. Thus, for example, the release of a target molecule used as a template during MIP synthesis is potentially challenging problem which may preclude practical use of the MIP even once, let alone re-use of the MIP.

For example, when a MIP is used to selectively bind an analyte in a sensor, removal of the bound analyte is generally problematic and, therefore, in some cases, the MIPs cannot be efficiently recycled. The remaining analyte in the MIP interferes with future attempts to use the same MIP to detect the analyte, thereby reducing the sensor's sensitivity for further use.

The present inventors have further envisioned that the abovementioned general limitation of MIPs can be overcome if the MIP can be induced to release a target molecule when desired, e.g., as a result of an external change.

While reducing the present invention to practice, the present inventors have developed a unique methodology for preparing molecularly imprinted smart polymers (MISPs) which undergo a physico-chemical change in response to an external change, such that a bound target molecule is released as a result of subjecting the MISP to the external change.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

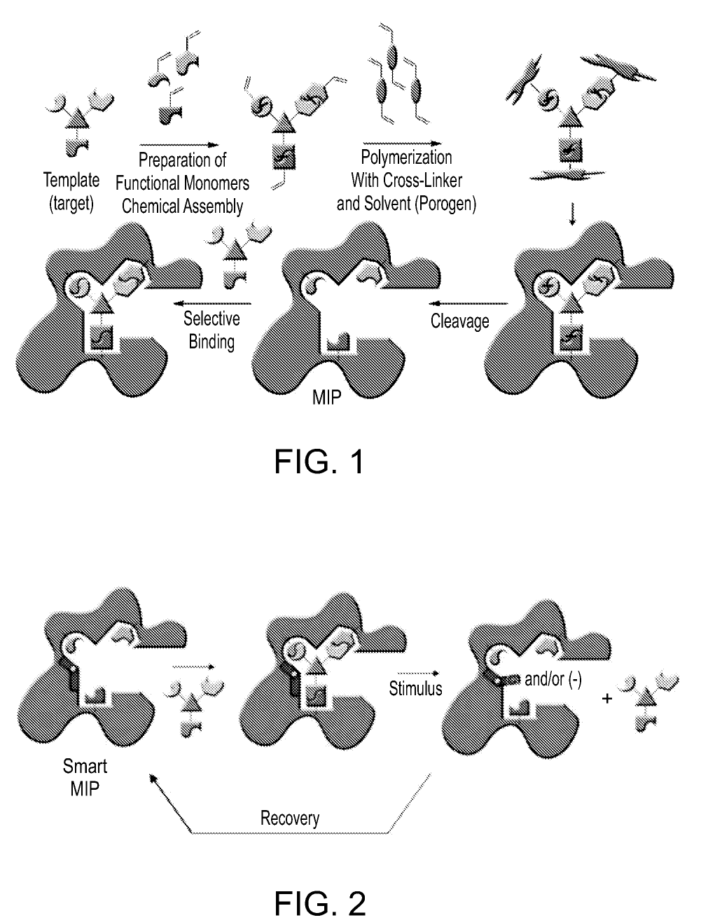

Referring now to the drawings, FIG. 1 depicts the preparation of a MIP. FIG. 2 depicts binding and release of a target molecule by a MISP, in accordance with embodiments of the present invention.

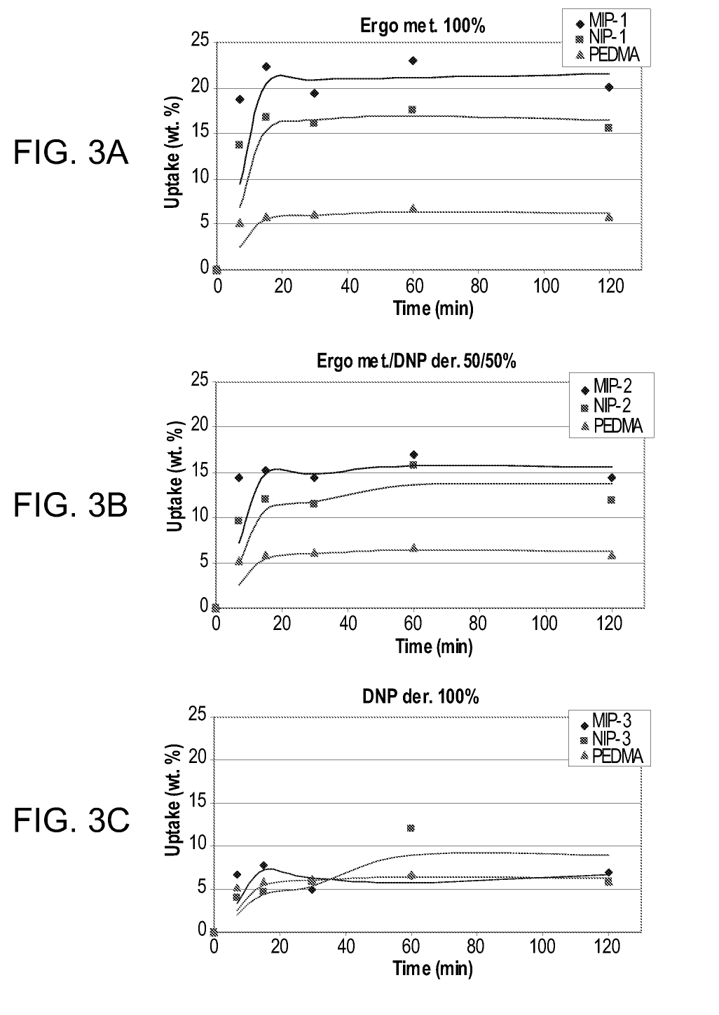

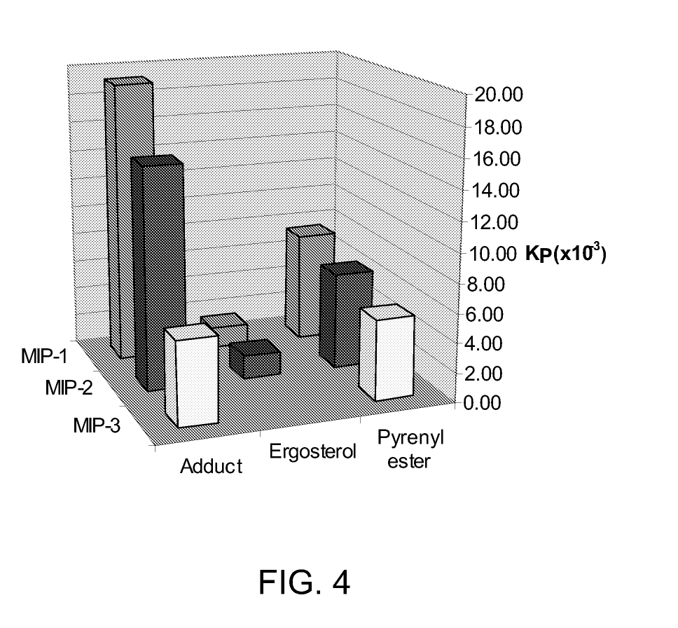

FIGS. 3A-3C and 4 show the effects of monomer type on binding of specific and non-specific a target molecule to a polymer.

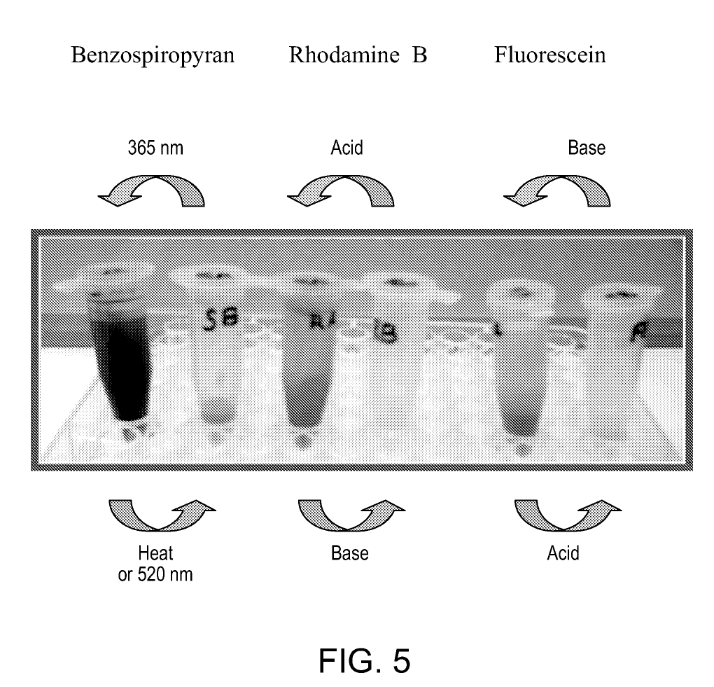

FIG. 5 shows samples of polymers prepared from exemplary responsive monomers, as well as transition of one form of the polymers to another as a result of an external change.

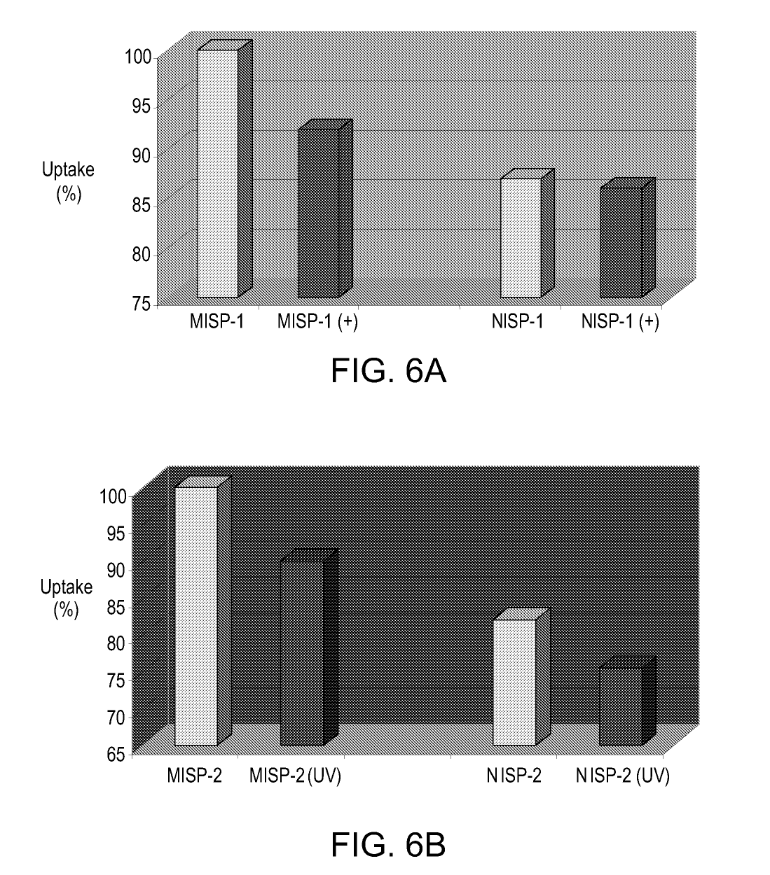

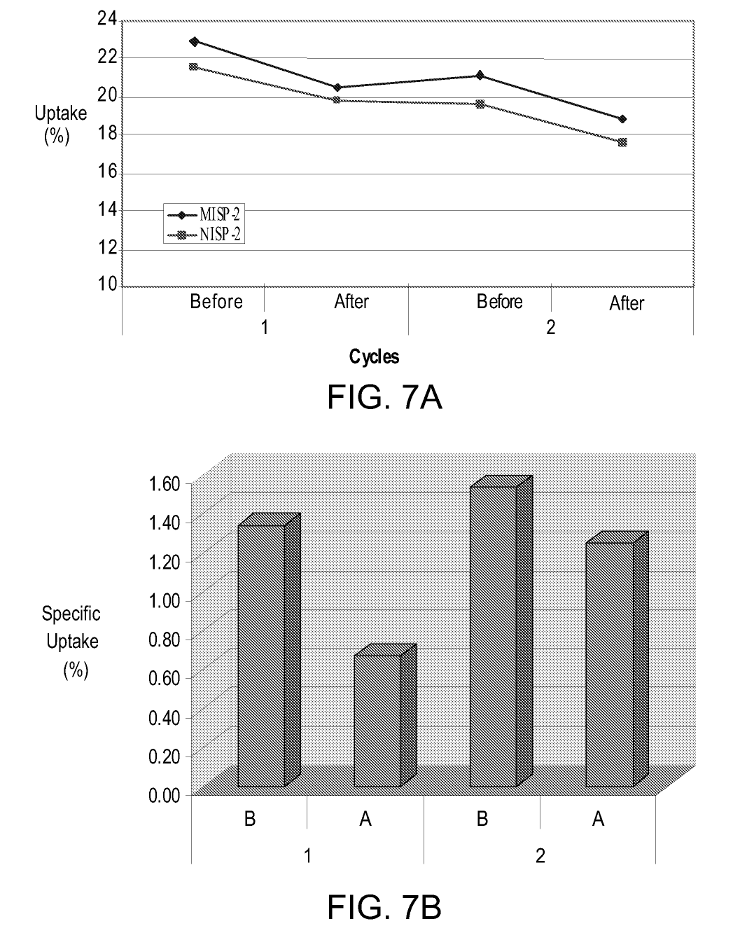

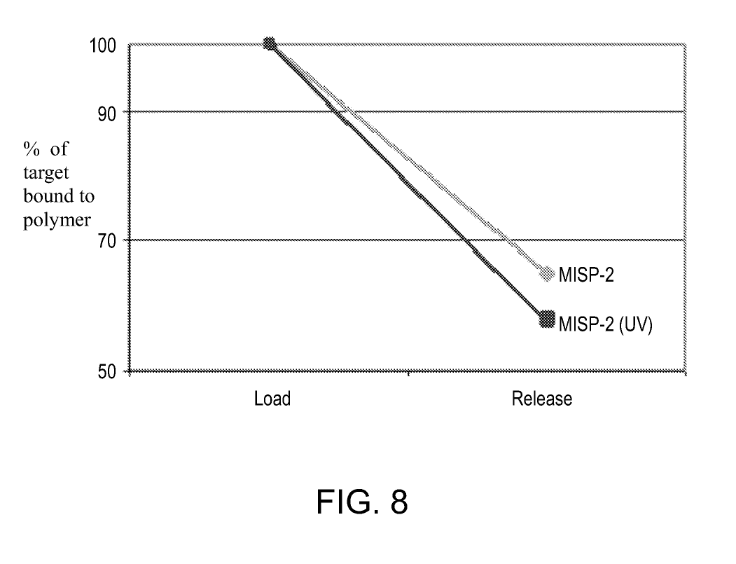

FIGS. 6A, 6B, 7A and 7B show uptake of a target molecule by exemplary polymers, as well as reduced uptake following activation of the polymers. FIG. 8 shows increased release of a target molecule by an exemplary polymer following activation.

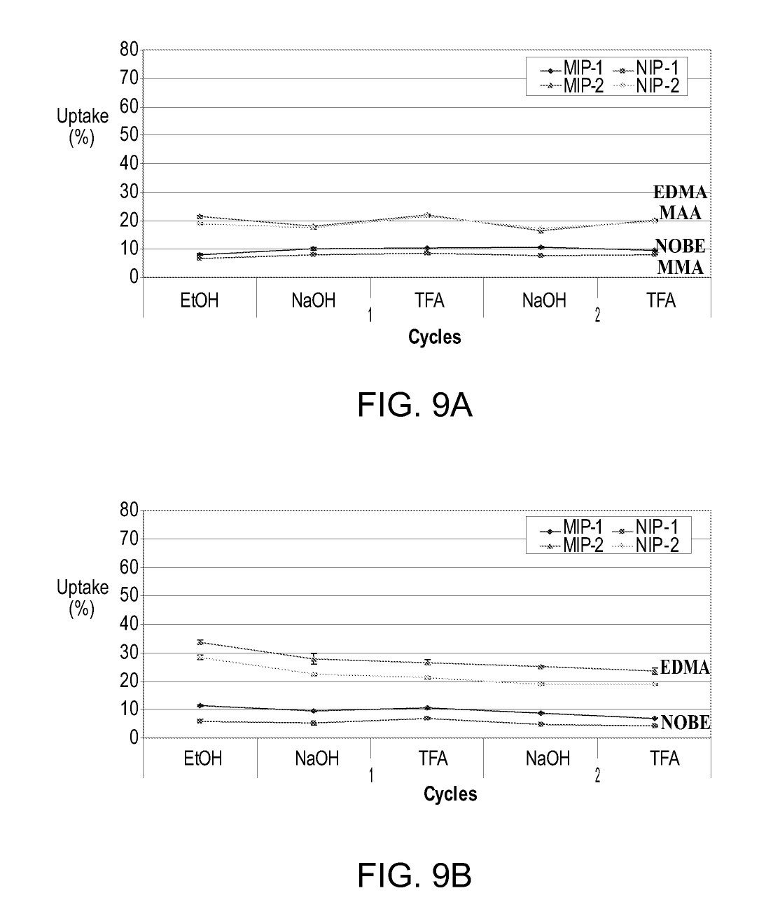

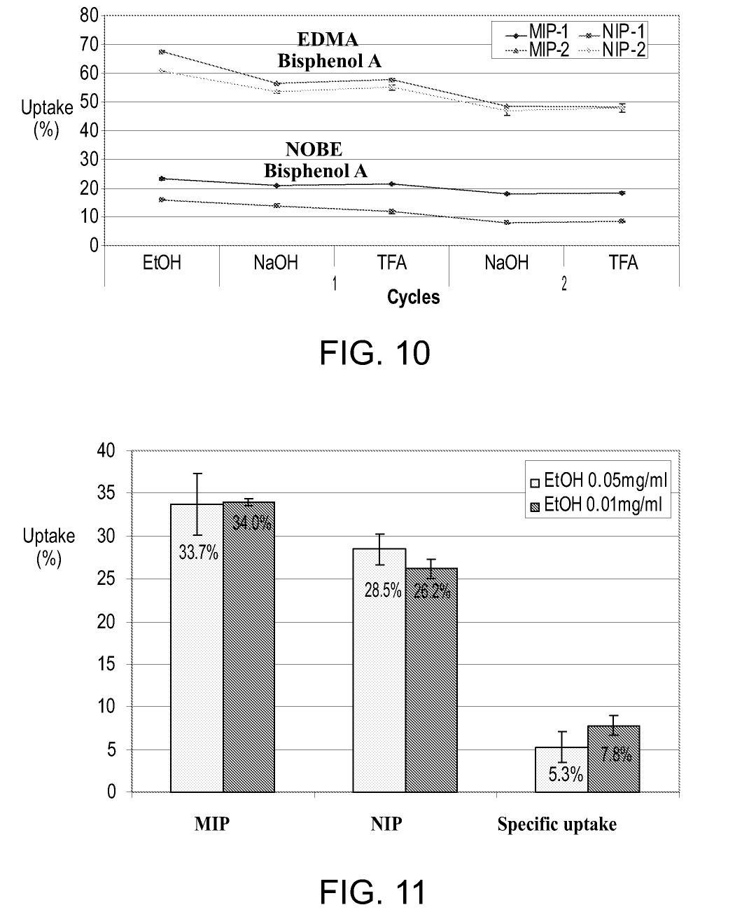

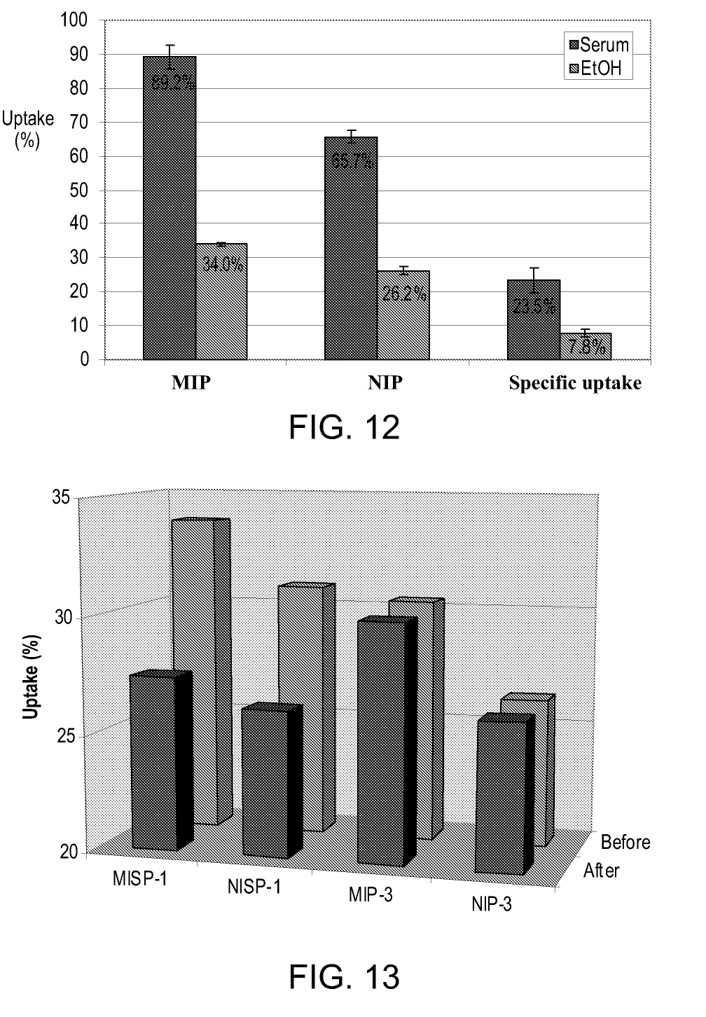

FIGS. 9A, 9B and 10 show effects of monomer type on binding of a target molecule by a polymer. FIGS. 11 and 12 shows effects of target concentration and solvent on specific and non-specific binding of a target molecule by a polymer.

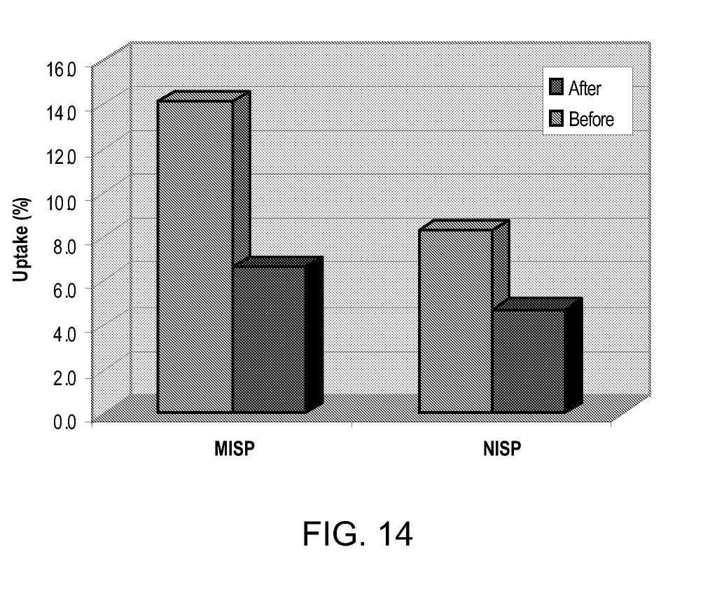

FIGS. 13 and 14 show uptake of a target molecule by exemplary polymers, as well as reduced uptake following activation of the polymers.

Accordingly, while reducing the present invention to practice, the present invention have designed and successfully prepared and practiced novel responsive monomers and MISPs obtained therefrom, which were shown to possess an advantageous “catch-and-release” mode of action, which allows using such MISPs for reversibly binding and releasing a target molecule, repeatedly.

According to an aspect of embodiments of the present invention, there is provided a molecularly imprinted polymer (MIP) comprising a plurality of monomeric units, wherein at least one of the monomeric units is a responsive monomeric unit being capable of undergoing a physico-chemical change in response to an external change. The responsive monomeric unit is incorporated within or attached to the molecularly imprinted polymer. The molecularly imprinted polymer is capable of selectively binding to a target molecule and releasing a bound target molecule in response to the external change. The MIP may comprise one or more species of monomeric unit. In some embodiments, the MIP comprises one or more species of monomeric unit other than the responsive monomeric unit(s).

In some embodiments, the plurality of monomeric units, both the responsive monomeric units and those other than the responsive monomeric units, composing the polymer, are linked to one another so as to form the polymeric backbone. In some embodiments, upon subjecting the polymer to an external change, a physico-chemical change in a responsive group within the responsive monomeric units is effected, as detailed hereinbelow, whereby this physico-chemical change results in selectively binding a target molecule, or in releasing a bound target molecule.

Compounds and moieties which are capable of undergoing a physico-chemical change in response to an external change are referred to herein as “smart” and/or “responsive”. Hence, the MIP of the above aspect of embodiments of the invention is also referred to herein as a “molecularly imprinted smart polymer” or “MISP”. Similarly, the abovementioned monomeric unit capable of undergoing a physico-chemical change in response to an external change is referred to as a “responsive monomeric unit”.

As used herein, the phrases “molecularly imprinted polymer” (also referred to as “MIP”) and “molecularly imprinted smart polymer” (also referred to as “MISP”) describe a polymer or smart polymer, respectively, which comprise regions having a structure (“imprint”) that is complementary to a target molecule and which are therefore capable of selectively binding the target molecule. The imprint can be characterized, for example, by a cavity in the polymer of a shape and size suitable for non-covalent binding to the target molecule and/or by functional groups positioned in a geometrical configuration suitable for binding complementary functional groups of the target molecule (i.e., shape and/or electrostatic complementarities).

In some embodiments, the MISPs described herein bind a target molecule via hydrophobic interactions, via covalent bonds or via electrostatic bonds, including hydrogen bonds.

The polymers can be linear, branched or cyclic, cross-linked or non-cross-linked, depending on their intended use.

The MIPs described herein can bind any of a wide variety of target molecules, as detailed hereinbelow. In some embodiments, the target molecule is of a molecular weight of up to 1,500 grams/mol.

As used herein, the phrase “physico-chemical change” describes a change in physical or chemical properties of a molecule, such as change in electric charge (e.g., generation or elimination of a positive or negative charge), change in polarity (e.g., generation or elimination of an electric dipole), change in conformation (e.g., isomerization), and/or change in configuration (e.g., formation and/or cleavage of one or more covalent bond(s)).

According to optional embodiments of the present invention, the physico-chemical change is selected from the group consisting of formation of a covalent bond linking two atoms in the responsive monomeric unit and cleavage of a covalent bond linking two atoms in the responsive monomeric unit. In some embodiments, neither of the two atoms is hydrogen.

In some embodiments, the formation and/or cleavage of a covalent bond is accompanied by a change in the electrostatic configuration in the molecule, namely, is accompanied by generation or elimination of a positive or negative charge and/or an electric dipole, at one or more positions of the molecule. Such a change includes also a change in a position or a direction of a charge or a dipole in the molecule.

Without being bound by any particular theory it is believed that formation or cleavage of a covalent bond, and optionally the corresponding electrostatic change in the molecule, results in a more significant event than other structural changes, such as cis-trans isomerization of a double bond. A formation and/or cleavage of a covalent bond is therefore potentially more useful for inducing release of a bound target molecule. Thus, in some embodiments, formation and/or cleavage of a covalent bond changes both the geometrical configuration (e.g., shape) of the monomeric unit, and the chemical properties of the monomeric unit (e.g., by creating and/or eliminating one or more functional groups, optionally together with generating or eliminating a charge or a dipole, as described herein). In contrast, cis-trans isomerization changes a geometrical conformation of a monomeric unit without altering the chemical properties of the chemical groups present.

It is further believed that formation or cleavage of a covalent bond results in a more significant, and therefore potentially more useful, change in molecular structure when neither of the two atoms is hydrogen.

In some embodiments, the physico-chemical change is reversible, for example, such that the MISP which changes from a first state to a second state in response to an external change can readily revert to the first state (e.g., when the external change is reversed and/or eliminated).

In some embodiments, the physico-chemical change involves a formation or cleavage of a covalent bond which forms or opens a ring (e.g., a cycloalkyl, a heteroalicyclic, a aryl, a heteroaryl, as defined herein).

Without being bound by any particular theory, it is believed that formation and/or opening of a ring is particularly suitable for a reversible physico-chemical change. For example, if a bond cleavage results in a formation of separate product, namely, a molecule that is not attached to, or being within, the polymer, the cleavage may effectively be irreversible if the product escapes from the system (e.g., by diffusion). In contrast, ring-opening bond cleavage does not generate a separate product, only a polymer with a different internal structure. Hence, ring-opening can be readily reversed by re-formation of the ring.

It is to be noted that, in some embodiments, the ring formed or opened in response to an external change, does not form a part of the polymeric backbone itself, but is rather attached to those moieties (polymerizable moieties, as detailed hereinbelow) that link the monomeric units to one another. Accordingly, the ring is composed of atoms that constitute a side chain of the polymeric backbone.

As used herein, the phrase “external change” describes a change which is external to the polymer, including, without limitation, a physical and/or chemical change in the environment of the polymer and/or a change in an external field that is applied to the polymer.

The “environment” of the polymer relates any medium where the polymer is placed in, including, but not limited to, a solution containing the polymer, a gas chamber containing the polymer, air, and the like. Physical and/or chemical changes in the environment include, but are not limited to, a presence of a chemical, a change in pH, a change in pressure, a change in temperature, exposure to a reducing agent, and exposure to an oxidizing agent. Accordingly, a formation or cleavage of a covalent bond in the responsive monomeric unit can be pH-dependent, temperature-dependent, pressure-dependent, and/or can be a result of oxidation, reduction or a chemical reaction.

A “change in an external field” encompasses any change as a result of application of or a change in the magnitude of, a field, including, but not limited to, exposure to (or removal of) sound waves, exposure to (or removal of) irradiation (e.g., visible light, ultraviolet light and/or infrared radiation), exposure to (or removal of) an electric current, exposure to (or removal of) a magnetic field, and/or a change in any of the above. Accordingly, a formation and/or cleavage of the covalent bond can be, for example, light-regulated, electrically-regulated, magnetically-regulated, etc.

According to some embodiments, the external change comprises a change in the environment, as described herein. In some embodiments, the external change is a chemical change, such as, for example, a presence of a chemical (including a change in concentration of a chemical) and a change in pH.

In some embodiments, the external change comprises a change in irradiation, such as exposure of the polymer, or of a solution containing the polymer, to irradiation.

In some embodiments, the change comprises both a chemical change, as above, and a change in the applied field (e.g., exposure to irradiation).

The phrase “external change”, according to embodiments of the invention, can be regarded as a trigger event that activates (or deactivates) the MISP by inducing the physico-chemical change described herein.

As used herein, the phrase “monomeric unit” refers to a unit in a polymer which can be viewed as a residue of a corresponding monomer (in analogy to e.g., amino acids in a peptide). Thus, for example, a unit of a formula —CH₂—C(R)H— (wherein R is any substituent) in a polymer is an exemplary monomeric unit of the polymer, and can optionally be obtained by polymerizing a corresponding monomer CH₂═CH—R.

In some embodiments, the responsive monomeric unit comprises at least one polymerizable moiety and a responsive moiety. The responsive moiety is capable of undergoing a physico-chemical change in response to the external change, thereby causing the monomeric unit to be a responsive monomeric unit. The polymerizable moiety forms a polymeric backbone of the polymer by linking the monomeric units of the abovementioned plurality of monomeric units to one another.

Optionally, the MISP (molecularly imprinted smart polymer) is formed from a plurality of responsive monomeric units, such that a polymeric backbone of the MISP is formed by linking the responsive monomeric units to one another (e.g., by linking the polymerizable moieties of the responsive monomeric units together).

Alternatively, the plurality of monomeric units of the MISP comprises both one or more responsive monomeric unit and a plurality of additional monomeric units, such that a polymeric backbone of the MISP is formed by linking the responsive monomeric unit(s) and the additional monomeric units to one another (e.g., forming a copolymer). The additional monomeric units optionally comprise at least one polymerizable moiety forming a polymeric backbone by linking the monomeric units to one another. In some embodiments, the polymerizable moiety of a responsive monomeric units and of other monomeric units is the same. Alternatively, the polymerizable moieties are different from one another, yet are linked to one another for forming the polymeric backbone.

As used herein, the phrase “polymerizable moiety” refers to a moiety suitable for forming a polymeric backbone, e.g., by linking to other polymerizable moieties, being the same or different. A wide variety of polymerizable moieties, as well as their chemical properties, will be familiar to the skilled artisan.

Representative example of suitable polymerizable moieties include, but are not limited to, vinyl, vinylphenyl (e.g., 4-vinylphenyl), 4-vinylbenzoate, itaconate, 1-vinylimidazole, 2-vinylpyridine, 4-vinylpyridine, 4-vinylimidazole, 4-vinylbenzyl-iminoacetate, acrylate, methacrylate, trifluoromethacrylate, acrylamide and methacrylamide. Monomeric units with such polymerizable moieties form polymeric backbones such as, for example, poly(styrene), poly(4-vinylbenzoate), poly(itaconate), poly(1-vinylimidazole), poly(2-vinylpyridine), poly(4-vinylpyridine), poly(4-vinylimidazole), poly(4-vinylbenzyl-iminoacetate), poly(acrylate), poly(methacrylate), poly(trifluoromethacrylate), poly(acrylamide), poly(methacrylamide) and copolymers thereof.

It is to be appreciated that the phrase “polymerizable moiety”, when used to describe a moiety in a polymer, refers to a moiety which is linked to at least two (typically two) adjacent moieties in the polymeric backbone (unless the polymerizable moiety is at a terminus of the backbone, in which case it is linked to one adjacent moiety in the backbone). Thus, for example, an acrylate moiety in a monomeric unit is —CH₂—C(CO₂—)H—, an acrylamide moiety is —CH₂—C(CO—NH—)H—, a methacrylate moiety is —CH₂—C(CO₂—)(CH₃)—, and a methacrylamide moiety is —CH₂—C(CO—NH—)(CH₃)—. Accordingly, a monomeric unit having one of the four aforementioned moieties will have a structure —CH₂—C(CO₂R)H—, —CH₂—C(CO—NHR)H—, —CH₂—C(CO₂R)(CH₃)— or —CH₂—C(CO—NHR)(CH₃)—, wherein R is any suitable moiety.

In contrast, when used to describe a moiety in a monomer, the phrase “polymerizable moiety” refers to a moiety suitable for being reacted so as to link to other monomers, rather than to a moiety which is already linked to other moieties. Thus, for example, an acrylate moiety in a monomer is CH₂═C(CO₂—)H, an acrylamide moiety is CH₂═C(CO—NH—)H, a methacrylate moiety is CH₂═C(CO₂—)CH₃, and a methacrylamide moiety is CH₂═C(CO—NH—)CH₃.

It is to be appreciated that many polymerizable moieties can be considered to comprise a smaller polymerizable moiety. Thus, for example, an acrylate moiety and an acrylamide moiety can each be considered to comprise an acryloyl moiety (—CH₂—C(CO—)H—), and a methacrylate moiety and an acrylamide moiety can each be considered to comprise a methacryloyl moiety (—CH₂—C(CO—)(CH₃)—).

According to optional embodiments of the present invention, at least a portion of the additional monomeric units (which are present in the MISP in addition to the responsive monomeric unit(s)) each comprise at least two polymerizable moieties. Monomeric units and monomers comprising two or more polymerizable moieties are referred to herein as “cross-linkers”.

Cross-linkers which may be used in embodiments of the present invention include, without limitation, cross-linkers in which two polymerizable moieties, as described herein, are linked by a —CH₂— group (e.g., N,N′-methylene-bisacrylamide), a —CH₂CH₂— group (e.g., ethylene dimethacrylate (EDMA), N,O-bismethacryloyl ethanolamine (NOBE)), a phenylene (—C₆H₄—) group (e.g., divinylbenzene, N,N′-phenylene-bisacrylamide), a pyridine linking group (e.g., 2,6-bisacrylamidopyridine), a bisphenol A linking group (e.g., bisphenol A dimethacrylate), as well as cross-linkers with more than two polymerizable moieties (e.g., trimethylolpropane trimethacrylate, i.e., CH₃CH₂C(—CH₂—O—C(═O)—C(═CH₂)CH₃)₃). Exemplary cross-linkers include EDMA, NOBE and bisphenol A dimethacrylate.

Without being bound by any particular theory, it is believed that cross-linkers are particularly suitable for inclusion in MIPs and MISPs because they increase a rigidity of the polymer, thereby preserving a molecular imprint in the polymer (e.g., by preventing elimination of the imprint due to movement of various components of the polymer). It is within the capabilities of the skilled artisan to select a proportion of cross-linker monomeric units to provide a suitable rigidity of the polymer.

According to another aspect of embodiments of the present invention, there is provided a process for producing a MISP as described herein, the process comprising polymerizing a plurality of monomers in the presence of a template molecule (e.g., in a mixture of the plurality of monomers and the template molecule), wherein at least one of the monomers is a responsive monomer being capable of undergoing a physico-chemical change (e.g., a physico-chemical change as described herein) in response to an external change as described herein.

Polymerization can be performed according to any suitable technique known in the art, including, but not limited to, free-radical polymerization, ring-opening polymerization, condensation, etc. For example, monomers comprising acryloyl and/or methacryloyl polymerizable moieties can be polymerized via free-radical polymerization (e.g., by adding an initiator of free-radical polymerization).

A “template molecule” is a molecule suitable for forming an imprinted polymer by having at least a similar, preferably identical, size, shape and functionalities, as the target molecule. In some embodiments, the template molecule is the target molecule. In some embodiments, a template molecule is a portion of target molecule which is used for e.g., recognizing the target molecule and/or binding the target molecule via that portion.

It is to be understood that the phrase “in the presence of a template molecule” is intended to encompass the presence of derivatives of the target molecule, for example, wherein the target molecule is covalently attached to a monomer so as to form a monomer having a moiety derived from the target molecule.

Optionally, the responsive monomer comprises at least one polymerizable moiety selected capable of linking to other monomers so as to form the polymer, and a responsive moiety selected capable of undergoing a physico-chemical change in response to the external change.

In some embodiments, at least some of the monomers are selected so as to have an affinity to the target molecule (and optionally and desirably to the template molecule). Examples of such selection include, without limitation, selecting a hydrophobic monomer to have an affinity to a hydrophobic target molecule, selecting a polar monomer to have an affinity to a complement polar target molecule, and selecting an ionic monomer to have an affinity to an ionic target molecule of the opposite charge. Selection is made according to the desired interaction between the resulting MISP and the target molecule, as described herein.

Polymerization is optionally performed in a solvent or porogen. A solvent in which polymerization is performed is optionally selected so as to facilitate the polymerization reaction, as well as to enhance the affinity of a monomer to a target molecule (e.g., selecting a polar solvent so as to enhance an affinity between a hydrophobic monomer and target, selecting a non-polar solvent to enhance an affinity between a polar monomer and target molecule). As a porogen the solvent induces a considerable polymer surface area, rendering maximum accessibility to the binding sites.

The solvent, or porogen, can be selected from a myriad of solvents, as long as it has the desired properties, as detailed herein.

The species of monomers to be polymerized will depend on the type of MISP which is being prepared. Thus, for example, a MISP comprising a responsive monomeric unit as well as at least one other type of monomeric units can be prepared by copolymerizing a responsive monomer and at least one other type of monomer. A MISP comprising a monomeric unit (a responsive monomeric unit and/or another monomeric unit) having a polymerizable moiety selected from the group consisting of acrylate, methacrylate, acrylamide and methacrylamide can be prepared by polymerizing a monomer having a polymerizable moiety selected from the group consisting of acrylate, methacrylate, acrylamide and methacrylamide, as described in further detail herein. A MISP comprising monomeric units having two polymerizable moieties (e.g., EDMA and/or NOBE monomeric units) in addition to a responsive monomeric unit can be prepared by copolymerizing the responsive monomeric unit with a monomer having two polymerizable moieties (e.g., the compounds EDMA and/or NOBE).

As exemplified hereinbelow, a variety of different types of responsive monomer can be used to prepare a MISP.

According to some embodiments of the invention, the responsive monomer can be represented as

A-L-B or

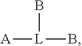

wherein A is a responsive moiety; B is a polymerizable moiety and L is absent or is a linker moiety.

In embodiments wherein L is absent, A-L-B is A-B, and

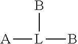

### is B-A-B or A-B-B.

As used herein, the term “linker” and the phrase “linking group” are used interchangeably and describe a group which attaches to a plurality of moieties, thereby linking the moieties together.

As used herein, the phrase “end group” describes a group with attaches to a single moiety.

In many embodiments, the structure of the linker moiety can be varied, provided it does not induce a deleterious chemical interaction (e.g., interfering with polymerization, interfering with the physico-chemical change of the responsive monomer). It is within the capabilities of a skilled artisan to select a suitable linker, or alternatively, to select a monomer in which L is absent, based on the aforementioned considerations, as well as other consideration such as cost and ease of synthesis.

Monomers having the structure A-L-B are referred to herein as “first generation” monomers. Monomers having the structure

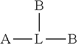

are referred to herein as “second generation” monomers. It is to be appreciated that second generation monomers are cross-linkers (monomers having two or more polymerizable groups) and are therefore particularly suitable for embodiments in which a high proportion of cross-linker is desired.

First generation and second generation monomers can be polymerized or copolymerized with one or more additional species of monomer to form a MISP by performing polymerization in the presence of a template molecule which is added thereto (e.g., forming a mixture of the monomer(s) and the target molecule).

Optionally, a MISP prepared using a first generation and/or second generation responsive monomer is prepared so as to provide a high likelihood that a responsive monomer is present in the MISP near a binding site for a target molecule, so as to enhance an ability of a physico-chemical change of the responsive monomer to induce release of a bound target molecule. In some embodiments, a proportion of responsive monomers is selected (e.g., based on a size of the target molecule and/or number of monomers bordering a binding site) to be high enough so as to result in a high statistical probability that at least one responsive monomeric unit borders a binding site. In some embodiments, responsive monomers are selected so as to have an affinity to the target molecule, so as to result in at least one responsive monomer being non-covalently bound to the template molecule during polymerization, thereby leading to at least one responsive monomeric unit being present at a binding site (imprint) for the target molecule in the MISP formed by polymerization.

According to optional embodiments, the responsive monomer comprises a moiety derived from the target molecule (in addition to a responsive moiety described herein and at least one polymerizable moiety described herein), wherein after the polymerizing, the moiety derived from the target molecule is released from the monomeric unit formed from the monomer by polymerization (e.g., by cleavage of a bond linking the target molecule moiety to the rest of the monomeric unit). Optionally, the moiety derived from a target molecule is attached to the rest of the monomer via a linking group with a readily cleavable bond (e.g., ester, carbonate, anhydride, mixed anhydride, ketal, imine, boronic ester, silyl ether, carbamate, and thioester), so as to facilitate release of the moiety.

Monomers comprising such a moiety derived from a target molecule are referred to herein as “third generation” monomers. An advantage of third generation monomers is that each third generation responsive monomer can create an adjacent binding site in the MISP, because the moiety derived from a target molecule serves as a template during polymerization, and a binding site is formed when the moiety is released. Consequently, each binding site in the MISP is bordered by at least one responsive monomeric unit.

Optionally, the moiety is attached via a —O—C(═O)—O— or —O—C(═O)—NH— group, such that the moiety derived from a target molecule comprises a —O—C(═O)—O— or —O—C(═O)—NH— group. A —O—C(═O)—O— group can optionally link a hydroxy group in a target molecule to a hydroxy group in a precursor of the monomer to form the monomer. A —O—C(═O)—NH— group can optionally link a hydroxy group of a target molecule with an amine group in a monomer precursor, or an amine group of a target molecule with a hydroxy group of a monomer precursor. Thus, the moiety derived from a target molecule optionally has a structure —O—C(═O)—O-T* or T*-O—C(═O)—NH— (wherein T*-OH is the target molecule, which is released along with CO₂ upon cleavage), or —O—C(═O)—NH-T* (wherein T*-NH₂ is the target molecule, which is released along with CO₂ upon cleavage).

Such groups can be further advantageous in that the C═O group therein separates the oxygen atoms (or oxygen atom and nitrogen atom) of the two hydroxy groups (or hydroxy group and amine group) being linked by a small distance, which mimics the small distance expected between the hydroxy groups (or hydroxy group and amine group) when an imprinted polymer binds non-covalently to a target molecule. It is thus believed that the —O—C(═O)—O— or —O—C(═O)—NH— group thereby results in more accurate molecular imprinting.

According to optional embodiments, the third generation responsive monomer described herein can be represented by:

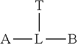

### or T-L-A-L-B

wherein A is said responsive moiety described herein, B is a polymerizable moiety described herein, L is absent or is a linker moiety, and T is a moiety derived from a target molecule.

In embodiments wherein at least one L is absent,

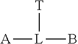

### is A-B-T or A-T-B or B-A-T, and T-L-A-L-B is T-A-B or T-A-L-B or T-L-A-B.

The linker moiety is optionally derived from a compound comprising at least three reactive functional groups (e.g., amine, hydroxy, thiohydroxy, carboxy, halide, oxo, oxirane, aziridine, carbonyl) suitable for forming linking groups. Examples include, without limitation, substituted aryl and heteroaryl, amino acids having a side chain which includes a reactive functional group.

Optionally, a linker moiety is a substituted (e.g., di-substituted or tri-substituted) alkyl, cycloalkyl, heteroalicyclic, aryl or heteroaryl group. In some embodiments, aryl or heteroaryl is attached to each of the moieties linked to the linker moiety by a linking group described herein, the linking group being a substituent of the aryl or heteroaryl. For example, an amine substituent optionally attaches to a moiety via an amine-derived linking group (e.g., amine linking group, amide linking group, urea linking group, carbamyl linking group, thiocarbamyl linking group, sulfonamide linking group). A hydroxy substituent optionally attaches to a moiety via a hydroxy-derived linking group (e.g., ether linking group, ester linking group, carbamyl linking group, silyl ether linking group). A carboxylic acid substituent optionally attaches to a moiety via a carboxylic acid-derived linking group (e.g., ester linking group, amide linking group, anhydride linking group).

In some embodiments, the linking moiety is an alkyl, an aryl, a cycloalkyl, a heteroalicyclic or a heteroaryl, substituted by two or more, preferably three or more, linking groups selected from the group consisting of alkoxy, aryloxy, amine, thioalkoxy, thioaryloxy, amide, carbonyl, carboxy, thiocarboxy, thiocarbonyl, sulfonate, sulfate, urea, disulfide, sulfonyl, sulfinyl, sulfonamide, hydrazine, carbamyl, thiocarbamyl and carbonate.

In an exemplary embodiment, the linker moiety is an aryl (e.g., phenyl) substituted by at least two of an amine, alkoxy, aryloxy, amide, carbamyl and or carbonate (e.g., a derivative of a compound substituted by an amine, a hydroxy and a carboxylic acid). A representative example is a linking moiety derived from 2-amino-5-hydroxybenzoic acid. The linker thus comprises a substituted benzene ring. The linker is optionally attached to other moieties via an amine-derived linking group, a hydroxy-derived linking group and a carboxylic acid derived linking group.

Optionally, the linking moiety is derived from an amino acid, thus having an amine, a carboxylic group and another group derived from the side chain of the amino acid. Such linking moieties can also be referred to as tri-substituted alkyl.

According to optional embodiments of the present invention, the responsive moiety (of a monomer and/or monomeric unit) comprises a heteroalicyclic ring, the heteroalicyclic ring comprising a responsive bond linking a carbon atom and a heteroatom (e.g., N, O and/or S), wherein the responsive bond cleaves in response to the external change described herein, such that the external change causes opening of said heteroalicyclic ring.

Without being bound by any particular theory, it is believed that such heteroalicyclic rings are particularly suitable for providing a physico-chemical change which is both readily reversible and which significantly alters the functionality of the monomer or monomeric unit, for example, by creating a new functional group (e.g., hydroxy, thiohydroxy, primary amine) and/or by changing a charge distribution (e.g., creating a negative charge on the abovementioned heteroatom and a positive charge elsewhere in the molecular structure).

In some embodiments, the carbon atom which is linked by the responsive bond is further linked to at least one electron donating moiety, such as, for example, a heteroatom (e.g., N, O, S, P) and/or an unsaturated bond. In exemplary embodiments, the carbon atom is linked to at least one electron donating moiety selected from the group consisting of an amine group and a conjugated pi-electron system. Optionally the pi-electron system includes a heteroatom, for example, nitrogen (e.g., —NR₂) at a position such that when the abovementioned carbon atom has a positive charge, the pi-electron system has a resonance form in which the positive charge is on the heteroatom (e.g., ═N⁺R₂)

According to optional embodiments, the responsive moiety has the general formula I:

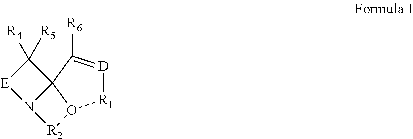

wherein:

the dashed lines denote that the oxygen atom is bound to either R₁ or R₂;

D is selected from the group consisting of N and CR₃;

E is an aromatic or heteroaromatic moiety, being substituted or non-substituted;

R₁ and R₂ are each independently selected from the group consisting of hydrogen, alkyl, alkenyl, alkynyl, cycloalkyl, heteroalicyclic, aryl, arylalkyl and heteroaryl, or R₁ attaches to R₆ to form a 5- or 6-membered cycloalkyl, heteroalicyclic, aromatic or heteroaromatic ring; and

R₃-R₆ are each independently selected from the group consisting of hydrogen, alkyl, alkenyl, alkynyl, cycloalkyl, heteroalicyclic, aryl, heteroaryl, arylalkyl, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl, or R₃ attaches to R₆ to form a 5- or 6-membered cycloalkyl, heteroalicyclic, aromatic or heteroaromatic ring, with the proviso that neither R₄ nor R₅ is hydrogen.

It is to be appreciated that whichever of R₁ and R₂ is bound to the oxygen atom depicted above will be a linking group. In addition, at least one of the substituents (e.g., any of R₁-R₆ and a substituent of the E moiety) is a linking group which attaches the responsive moiety to another moiety in the monomer or monomeric unit (e.g., a polymerizable moiety, a linker moiety). The other substituents are end groups, as this phrase is defined herein, except wherein the E moiety comprises a linking group substituent which attaches to more than one position of the aromatic or heteroaromatic moiety.

Optionally, the E moiety is attached to neighboring atoms (e.g., the N atom and the carbon atom substituted by R₄ and R₅) at adjacent positions of the E moiety, such that the E moiety comprises an aromatic or heteroaromatic ring which is fused to a 5-atom heteroalicyclic ring which includes the N atom depicted in Formula I and 2 atoms of the E moiety.

In some embodiments, E is phenylene, that is, an aryl linking group containing a benzene ring (i.e., —C₆H₄—).

In some embodiments, R₄ and R₅ are each a C₁₋₄ alkyl (i.e., an alkyl group containing 1-4 carbon atoms). Optionally, the alkyl is non-substituted. Optionally, R₄ and R₅ are each methyl.

In some embodiments, R₆ is hydrogen.

In some embodiments, R₁ is selected from the group consisting of a substituted or non-substituted aryl and a substituted or non-substituted heteroaryl.

Optionally R₁ (and not R₂) is covalently bound to the O atom depicted in Formula I. In some embodiments, R₁ comprises two carbon atoms linked by a double bond (e.g., wherein R₁ is aryl or heteroaryl), wherein one carbon atom is linked to the D moiety and the other carbon atom is linked to the O atom. In such embodiments, the heteroalicyclic ring comprising D, R₁ and the O atom is a pyran ring if D is CR₃ and an oxazine ring if D is N. In exemplary embodiments, the heteroalicyclic ring is pyran, and the compound is referred to as a spiropyran derivative. In other exemplary embodiments, the ring is oxazine, and the compound is referred to as a spirooxazine derivative. Exemplary spiropyran derivatives wherein R₁ is an aryl comprising a substituted or non-substituted benzene ring (e.g., phenylene and substituted derivatives thereof) are referred to herein as benzospiropyran derivatives.

In such embodiments wherein R₂ is not bound to the O atom, R₂ is optionally a C₁₋₄ alkyl group or suitably substituted group.

In some embodiments, D is ═CH—. In exemplary embodiments, R₁ is an aryl linking group, such as nitrophenylene (e.g., wherein the nitro group is at a para position with respect to the O atom of the pyran ring). Optionally R₂ is an alkyl (e.g., methyl) end group. Alternatively, R₂ is an alkyl linking group (e.g., —CH₂CH₂—) which links to the polymerizable moiety (e.g., methacrylate). Optionally, the alkyl linking group links to one or more polymerizable moieties (e.g., methacrylamide) via a linking group which is a substituent of the alkyl (e.g, —N(CH₂CH₂—)₂)

In some embodiments, D is N. In exemplary embodiments, R₁ is naphthylene, that is, an aryl linking group derived from napthalene (i.e., —C₁₀H₈—) or substituted naphathalene. Optionally, the naphthylene is linked to a polymerizable moiety (e.g., methacrylamide), optionally via a linking group which is a substituent of the naphthylene. In an exemplary embodiment, R₂ is alkyl (e.g., methyl).

Alternatively to the above, R₂ (and not R₁) is covalently bound to the O atom depicted in Formula I. Optionally, R₂ is an alkyl linking group (e.g., —CH₂CH₂—).

In some embodiments, R₂ is an arylalkyl linking group which is linked to the oxygen an nitrogen atoms depicted in Formula I. Optionally, the aryl group in an arylalkyl group is bound to the oxygen and the alkyl group is bound to the nitrogen. Alternatively, the alkyl group in an arylalkyl group is bound to the oxygen and the aryl group is bound to the nitrogen.

In some embodiments, D is ═CH—. Optionally, R₁ is selected from the group consisting of substituted or non-substituted aryl and substituted or non-substituted heteroaryl. In exemplary embodiments, R₁ is nitrophenyl (e.g., p-nitrophenyl). Optionally, E is a benzene ring that is attached to the polymerizable moiety (e.g., methacrylamine).

Optionally, R₃ and R₆ are joined so as to form a cycloalkyl, heteroalicyclic, aryl or heteroaryl ring (e.g., a benzene ring) which includes R₃, R₆ and D.

According to further optional embodiments, the responsive moiety has the general formula II:

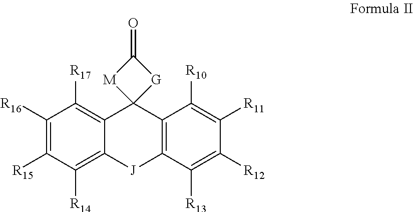

wherein:

G is selected from the group consisting of O, S and NR₁₉;

J is selected from the group consisting of O, S and NR₁₈;

M is an aromatic or heteroaromatic moiety, being substituted or non-substituted; and

R₁₀-R₁₇ are each independently selected from the group consisting of hydrogen, alkyl, alkenyl, alkynyl, cycloalkyl, heteroalicyclic, aryl, heteroaryl, arylalkyl, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl; and

R₁₈ and R₁₉ are each independently selected from the group consisting of hydrogen, alkyl, alkenyl, alkynyl, cycloalkyl, heteroalicyclic, aryl and heteroaryl.

Optionally, the M moiety is attached to neighboring atoms (e.g., the carbon atom of the C═O group and the carbon atom of the tricyclic moiety depicted in Formula II) at adjacent positions of the M moiety, such that the M moiety comprises an aromatic or heteroaromatic ring which is fused to a 5-atom heteroalicyclic ring which includes the G moiety depicted in Formula I and 2 atoms of the M moiety.

It is to be appreciated that at least one of the substituents (e.g., any of R₁₀-R₁₉ and a substituent of the M moiety) is a linking group which attaches the responsive moiety to another moiety in the monomer or monomeric unit (e.g., a polymerizable moiety, a linker moiety). The other substituents are end groups, as this phrase is defined herein, except wherein the M moiety comprises a linking group substituent which attaches to more than one position of the aromatic or heteroaromatic moiety.

In exemplary embodiments, M is phenylene (—C₆H₄—).

In exemplary embodiments, R₁₀, R₁₁, R₁₃, R₁₄, R₁₆ and R₁₇ are each hydrogen.

In exemplary embodiments, J is oxygen.

In some embodiments, at least one of R₁₂ and R₁₅ is selected from the group consisting of hydroxy, thiohydroxy and amine.

Optionally, G is NR₁₉ and R₁₉ is alkyl. The R₁₉ alkyl is optionally a linking group linked to at least one polymerizable moiety. In some embodiments, alkyl linking group is linked directly to a polymerizable moiety (e.g., methacrylate). In some embodiments, the alkyl linking group is linked to at least one polymerizable group via an additional linking group, such as —N(CH₂CH₂—)₂, —NH—C(═O)—C(NH—)H—(CH₂)₄—NH— (a lysine linking group), or a 2-amino-5-hydroxybenzoic acid derivative described herein. In some embodiments, the In exemplary embodiments, R₁₂ and R₁₅ are each an amine group, for example, dialkylamine (e.g., —N(CH₂CH₂)).

Optionally, G is oxygen.

In exemplary embodiments, R₁₂ and R₁₅ are each hydroxy. M is optionally a benzene ring that is attached to a polymerizable moiety (e.g., methacrylamide).

In additional exemplary embodiments, R₁₂ is hydroxy and R₁₅ is linked to a polymerizable moiety. R₁₅ is optionally a hydroxy-derived linking group (e.g., alkoxy, aryloxy, carboxy, carbamyl), for example, aryloxy. In exemplary embodiments, R₁₅ is —O—(C₆F₄)—C(═O)—, wherein C₆F₄ is a fluorinated phenylene linking group.

According to further optional embodiments, the responsive moiety is a triarylmethane derivative, comprising methane substituted by three (optionally substituted) aryl or heteroaryl groups. Triarylmethane derivatives are characterized in that the methane moiety has a form with a stable carbocation, wherein the methane moiety is not bound to any moiety besides the three aryl or heteroaryl groups. The carbocation can bind to a stable anion (e.g., OH⁻, halide, —CO₂⁻, —SO₃⁻). The responsive moiety is linked to the rest of the monomer or monomeric unit via a substituent of one of the aryl or heteroaryl groups.

Optionally the responsive moiety has the general formula III:

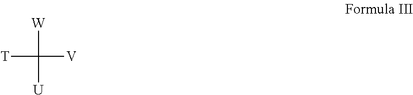

wherein:

T, U and V are each independently selected from the group consisting of substituted or non-substituted aromatic moiety and substituted or non-substituted heteroaromatic moiety; and

W is selected from the group consisting of hydroxy, halide, carboxy, and sulfonate (e.g., hydroxy).

The responsive moiety is linked to the rest of the monomer or monomeric unit via a substituent of at least one of T, U and V. In some embodiments, W is a linking group (e.g., carboxy, sulfonate) which links to any one of T, U and V, i.e., W is a substituent of T, U or V.

Optionally, each of T, U and V is a substituted or non-substituted phenyl group.

Optional responsive moieties include derivatives of malachite green, bromocresol green, bromocresol purple and bromothymol blue, wherein the malachite green, bromocresol green, bromocresol purple and bromothymol blue are linked to the rest of the monomer or monomeric unit via a substituent of one of the aryl groups thereof.

It is to be understood, that the above-described structures of responsive moieties are intended to encompass both embodiments wherein the described structure is a structure of the responsive moiety before undergoing a physico-chemical change, and embodiments wherein the described structure is a structure of the responsive moiety after undergoing a physico-chemical change.

The above-described structures of responsive moieties are intended to encompass embodiments wherein the described structure is a part of a responsive monomer (prior to forming a MISP) and is a part of a responsive monomeric unit that is comprised within the MISP.

The present embodiments encompass monomers formed from any combination of responsive moieties, polymerizable moieties and optionally linking moieties, as described herein, as long as such a combination is feasible.

The present embodiments further encompass MISPs formed from any combination of responsive monomers, other monomers and any cross-linkers, as described herein, as long as such a combination is feasible.

Accordingly, according to another aspect of embodiments of the invention, there is provided a library of responsive monomers, as described herein. Such a library can be used for forming a variety of MISPs, as described herein, by selecting a monomer which is particularly suitable for preparing a MISP for a selected target molecule. The monomer may be selected, for example, based on a known affinity to the target molecule, a predicted affinity to a template (e.g., predicted based on structure, polarity and/or functional groups of the monomer and template), results from previous MIP preparations, its response to a pre-determined external change and/or routine preliminary experimentation.

As used herein throughout, the term “alkyl” refers to a saturated or unsaturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. When substituted, the substituent group can be, for example, alkenyl, alkynyl, cycloalkyl, heteroalicyclic, aryl, heteroaryl, oxo, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl, as these terms are defined herein. The alkyl may be a linking group or an end group, as these terms are defined herein.

As used herein throughout, the term “alkenyl” refers to a substituted or non-substituted unsaturated aliphatic hydrocarbon having an unsaturated double bond.

As used herein throughout, the term “alkenyl” refers to a substituted or non-substituted unsaturated aliphatic hydrocarbon having an unsaturated triple bond.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene and adamantane. A cycloalkyl group may be substituted or unsubstituted. When substituted, the substituent group can be, for example, alkyl, alkenyl, alkynyl, heteroalicyclic, aryl, heteroaryl, oxo, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl, as these terms are defined herein. The cycloalkyl may be a linking group or an end group, as these terms are defined herein.

An “aryl” group or “aromatic moiety” refers to an all-carbon monocyclic or fused-ring polycyclic (i.e., rings which share adjacent pairs of carbon atoms) group having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, alkenyl, alkynyl, cycloalkyl, heteroalicyclic, heteroaryl, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl, as these terms are defined herein. The aryl may be a linking group or an end group, as these terms are defined herein.

A “heteroaryl” group or “heteroaromatic moiety” refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, alkenyl, alkynyl, cycloalkyl, heteroalicyclic, aryl, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl, as these terms are defined herein.

The heteroaryl may be a linking group or an end group, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, lone pair electrons, alkyl, alkenyl, alkynyl, cycloalkyl, aryl, heteroaryl, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl, as these terms are defined herein. Representative examples are 4,5-dihydroimidazole, piperidine, piperazine, tetrahydrofuran, tetrahydropyran, morpholine and the like. The heteroaryl may be a linking group or an end group, as these terms are defined herein.

As used herein, the terms “amine” and “amino” refer to a —NR′R″ group, wherein R′ and R″ are selected from the group consisting of hydrogen and substituted or unsubstituted alkyl, cycloalkyl, heteroalicyclic (bonded through a ring carbon), aryl, and heteroaryl (bonded through a ring carbon), wherein when the amino group is a linking group, at least one of R′ and R″ is absent, such that the group is, e.g., a —N(R′)— group. Optionally, R′ and R″ are selected from the group consisting of hydrogen and alkyl comprising 1 to 4 carbon atoms. Optionally, R′ and R″ are each hydrogen.

A “hydroxy” group refers to an —OH group.

An “alkoxy” group refers to both an —O-alkyl end group and an —O-cycloalkyl end group, as well as to —O-alkylene- and —O-cycloalkyl- linking groups, as defined herein.

An “aryloxy” group refers to both an —O-aryl end group and an —O-heteroaryl end group, as well as to —O-aryl- and —O-heteroaryl- linking groups, as defined herein.

It is to be appreciated that an alkoxy or aryloxy group is a linking group whenever the alkyl, cycloalkyl, aryl or heteroaryl group therein is a linking group.

An “ether” refers to an alkoxy or aryloxy group, wherein the oxygen atom of the alkoxy or aryloxy group is linked to an alkyl, cycloalkyl, heteroalicyclic (through a ring carbon), aryl or heteroaryl (through a ring carbon).

A “thiohydroxy” group refers to a —SH group.

A “thioalkoxy” group refers to both an —S-alkyl end group, and an —S-cycloalkyl end group, as well as to —S-alkylene- and —S-cycloalkyl- linking groups, as defined herein.

A “thioaryloxy” group refers to both an —S-aryl and an —S-heteroaryl end group, as well as to —S-aryl- and —S-heteroaryl- linking groups, as defined herein.

A “sulfide” refers to both a thioalkoxy and a thioaryloxy end group, wherein the group is linked to an alkyl, cycloalkyl, aryl, heteroaryl or heteroalicyclic group.

A “disulfide” group refers to both a —S-thioalkoxy and a —S-thioaryloxy group.

An “arylalkyl” group refers to a -alkyl-aryl or -aryl-alkyl end group, and to an -alkyl-aryl- linking group.

A “carbonyl” group refers to a —C(═O)—R′ end group, where R′ is defined as hereinabove, and to a —C(═O)— linking group.

A “thiocarbonyl” group refers to a —C(═S)—R′ end group, where R′ is as defined herein, and to a —C(═S)— linking group.

An “oxo” group refers to a ═O group.

A “carboxy” or “carboxylate” encompasses both —C(═O)—O—R′ and R′C(═O)—O— end groups, and a —C(═O)—O— linking group, as defined herein.

A “carboxylic acid” group refers to a —C(═O)—OH group.

An “ester” refers to a carboxylate end group wherein R′ is not hydrogen, and to a carboxylate linking group wherein the oxygen atom of the carboxylate is linked to an alkyl, cycloalkyl, heteroalicyclic (through a ring carbon), aryl or heteroaryl (through a ring carbon)

A “thiocarboxy” or “thiocarboxylate” group refers to both —C(═S)—O—R′ and —O—C(═S)R′ end groups, as well as to a —C(═S)—O— linking group.

A “halo” or “halide” group refers to fluorine, chlorine, bromine or iodine.

A “sulfinyl” group refers to an —S(═O)—R′ end group, where R′ is as defined herein, and to a —S(═O)— linking group.

A “sulfonyl” group refers to an —S(═O)₂—R′ end group, where R′ is as defined herein, and to a —S(═O)₂— linking group.

A “sulfonate” group refers to an —S(═O)₂—O—R′ end group, where R′ is as defined herein, and to an —S(═O)₂—O— linking group.

A “sulfate” group refers to an —O—S(═O)₂—O—R′ end group, where R′ is as defined as herein, and to a —O—S(═O)₂—O— linking group.

A “sulfonamide” or “sulfonamido” group encompasses both —S(═O)₂—NR′R″ and R′S(═O)₂—N(R′)— end groups, and a —S(═O)₂—N(R′)— linking group, as defined herein.

A “carbamyl” or “carbamate” group encompasses —OC(═O)—NR′R″ and R′OC(═O)—NR″— end groups and a —OC(═O)—NR″— linking group.

A “thiocarbamyl” or “thiocarbamate” group encompasses —OC(═S)—NR′R″ and R′OC(═S)—NR″— end groups and a —OC(═S)—NR″— linking group.

An “amide” or “amido” group encompasses —C(═O)—NR′R″ and R′C(═O)—NR″— end groups and a —C(═O)—NR″— linking group.

A “urea” group refers to an —N(R′)—C(═O)—NR″R′″ end group, where each of R′ and R″ is as defined herein, and R′″ is defined as R′ and R″ are defined herein, and to an —N(R′)—C(═O)—NR″— linking group.

An “imine” group refers to a ═N—R′ end group or ═N— linking group.

A “ketal” group refers to a —O—C(R′)(R″)—O—R′″ end group or to a —O—C(R′)(R″)—O— linking group.

A “boronic ester” refers to a —O—B(R′)—OR″ end group or to a —O—B(R′)—O— linking group.

As used herein, “silyl ether” refers to a —O—Si(R′)(R″)—O— linking group.

A “nitro” group refers to an —NO₂ group.

A “nitrile” group refers to a —C≡N group.

The term “isonitrile” describes a —N≡C group.

The term “nitroso” describes a —O—N═O group.

The term “hydrazine”, as used herein, describes a —NR′—NR″R′″ end group or a —NR′—NR″— linking group, as these phrases are defined hereinabove, wherein R′ and R″ are as defined herein, and R′″ is as defined herein for R′ and R″.

As used herein, the term “epoxide” describes a

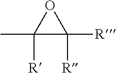

end group or a

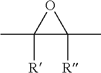

linking group, as these phrases are defined hereinabove, where R′, R″ and R′″ are as defined herein.

As used herein, the term “thiirane” describes a group that is equivalent to an epoxide, wherein the oxygen atom of the epoxide is replaced with a sulfur atom.

As used herein, the term “aziridine” describes a group that is equivalent to an epoxide, wherein the oxygen atom of the epoxide is replaced with a nitrogen atom, and the nitrogen atom binds, in addition to two adjacent carbon atoms, R″″, wherein R″″ is defined according to the same definition as R′ and R″.

According to another aspect of embodiments of the present invention, there is provided a MIP (e.g., a MISP) produced according to any process described herein.

According to optional embodiments, a molecularly imprinted polymer described herein is for reversibly binding a target molecule of the molecularly imprinted polymer (e.g., a target molecule used to imprint the polymer during polymerization).

Thus, according to a further aspect of embodiments of the present invention, there is provided a use of a MIP described herein for reversibly binding a target molecule of the MIP.

According to an aspect of the present invention, there is provided a method of selectively and reversibly binding a target molecule, the method comprising contacting a MIP (e.g., a MISP) described herein with the target molecule.

As exemplified hereinbelow, MISPs prepared according to embodiments of the invention exhibit selective binding to a target molecule, and the binding is reversed by an external change (also referred to herein as “activation” of a polymer), such as a change in pH and/or light (e.g., visible light, ultraviolet light). As further exemplified therein, the MISPs can be returned to the original state, which selectively binds a target molecule, by reversing the external change (e.g., returning a pH to the original value or a similar value) or by an additional external change (e.g., light or thermal).

It is within the capabilities of a skilled artisan to determine an external change suitable for a particular MISP, using, for example, readily determined properties of the MISP (e.g., absorption spectra of a responsive moiety, for activation by light), apparent protonated and deprotonated forms of a responsive moiety (e.g., for predicting an effect of a pH change), as well as the Examples hereinbelow, wherein activation of a variety of MISPs is demonstrated, including MISPs with a responsive moiety according to Formula I (e.g., benzospiropyran derivatives, indolenine derivatives) and MISPs with a responsive moiety according to Formula II (e.g., fluorescein derivatives, Rhodamine B derivatives).

In some embodiments, the monomer or monomeric unit exhibit photochromism (i.e., a color change resulting from exposure to light) and/or halochromism (i.e., a color change resulting from a change in pH). As exemplified hereinbelow, such properties are useful indicators of a physico-chemical change and an external change which causes it.

The MISPs described herein may be used for any use and/or method known in the art for which a MIP is useful. Examples include, without limitation, for selective binding in sensors (e.g., sol-gel based sensors), solid phase separation, removal of unwanted (e.g., toxic) materials, in diagnostic applications, as pharmaceuticals and as drug delivery carriers, as is further detailed hereinbelow. The MISPs described herein are advantageous for such applications, as the polymer can be reused after removing bound target molecules by inducing a physico-chemical change. Additional uses of MIPs, including uses in which the ability to reuse the MIP is advantageous, will be apparent to those of skill in the art.

In exemplary embodiments, the MISP is used to selectively bind a target molecule which is a biological or non-biological marker. For example, utilization of a rapid diagnostic device for systemic fungal infection by selectively binding a fluorescent adduct of ergosterol (e.g., the ergosterol-triazolinone-pyrene adduct) by a MIP is described in International Patent Application No. PCT/IL2006/001318 (Publication No. WO/2007/057891), which can be improved by using a reusable MISP according to embodiments of the present invention.

Additional exemplary target molecules include, but are not limited to, peptides, oligopeptides, and a polypeptides, including, for example, hormones, co-enzymes and peptidic drugs, amino acids (both naturally occurring or modified), drugs (including, for example, antibiotics, anti-proliferative agents, anti-inflammatory agents, psychotropic drugs, steroids, and any other drugs), biological markers, radioactive agents, pesticides, explosives, carbohydrates, nucleotides, including oligonucleotides, polynucleotides, anti-senses, and the like, and other chemical reagents.

In some embodiments, the MISP is used to selectively bind a portion of target molecule.

Application for which the MISPs maybe used include, for example, as mimics of enzymes and catalytic antibodies (e.g., by using template which mimics a transition state of a reaction), as biomimetic receptors, in diagnostic kits, in immunoassays, in drug delivery, as drug carriers that can bind a drug and release it is response to an external change, in high-throughput screening for e.g., drugs such as inhibitors, ligands, etc., as sensors (e.g., for selective detection, monitoring of target compounds), in organic synthesis (e.g., as microreactors, as selective protecting groups and/or selective scavengers), and in separations (e.g., solid phase extraction and chromatography).

In some embodiments, a sensor comprises a thin layer of MIP on a substrate (e.g., silica), and the sensor detects the presence of a target molecule bound to the MIP by determining an increase in mass (e.g., by quartz crystal microbalance) of the MIP on the surface of the substrate.

General guidelines for determining if a MISP as described herein is suitable for an intended use include, but are not limited to, its response to an external change, a nature of the physico-chemical change is response to the external change which is reversible (although this is not a pre-requisite for some applications), a binding of the MISP to the target molecule (or a portion thereof) which is comparable to that of a corresponding MIP, upon subjecting the MISP to an external change, as described herein, a reduced binding to the target and/or template molecule, and accordingly, an efficient release of, a target and/or template molecule, as described herein (more efficient than in the case of a corresponding MIP), and a binding and release of a target molecule and/or a template molecule which are reversible to the extent that more than 1, more than 2, more than 3, more than 4, etc., and even more than 10 or more than 20 cycles of binding and release can be performed with the same MISP.

The above can be determined using methods known in the art. Exemplary such methods are described in the Examples section that follows.

By “reduced binding” it is meant that upon subjecting the MISP to an external change, the binding to target or template molecule is reduced by at least 10%, at least 20%, at least 30%, at least 40%, at least 50%, at least 60%, at least 70%, at least 80%, at least 90%, and even by 100%. In some embodiments, binding is reduced by at least 50% or at least 70%.

Any of the MISPs described herein can be packaged in a packaging material and identified in print, in or on the packaging material, for use for reversible binding an indicated target molecule or a family of target molecules, and/or for use in an intended application that benefits from the reversible binding, as described herein.

Any of the responsive monomers described herein can also be packaged in a packaging material and identified in print, in or on the packaging material, for use in preparing a corresponding MISP, optionally while indicating an intended use of the MISP.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods

### Materials:

Ergosterol-triazolinedione-pyrene (Erg-TAD-Py) was prepared according to the procedure described in International Patent Application No. PCT/IL2006/001318 (Publication No. WO/2007/057891).

Ergosterol methacrylate was prepared by reacting ergosterol with methacryloyl chloride in the presence of n-butyllithium at −78° C. (in tetrahydrofuran).

2-(2,4-dinitrophenylamino)ethyl methacrylate (DNP) was prepared by reacting 2,4-dinitroaniline with 2-bromoethanol followed by methacryloyl chloride.

Ethyl 4-(pyren-1-yl)butanoate was prepared by reacting 4-(pyren-1-yl)butanoic acid with thionyl chloride (SOCl₂) and ethanol.

NOBE (N,O-bismethacryloyl ethanolamine) was prepared by cooling a solution of 2.33 grams methacryloyl chloride in 20 ml dichloromethane to a temperature of −30° C. under N₂, and adding dropwise a solution of 0.70 grams ethanolamine and 3.02 grams ethyl diisopropylamine in 20 ml dichloromethane. The reaction mixture was stirred for 2 hours and the mixture was then allowed to return to room temperature. An aqueous solution of 20% HCl was added, the organic phase was separated and washed twice with water and twice with brine, dried over anhydrous sodium sulfate, and the solvent was removed under reduced pressure. The crude oil was purified by CombiFlash® chromatography using linear gradient EA in PE (yield was 34%).

Bisphenol A dimethacrylate was prepared by reacting bisphenol A with methacryloyl chloride (in dichloromethane).

Inactivated fetal calf serum was obtained from Biological Industries (Israel).

2-Iodoethanol was obtained from Aldrich, or by reacting 2-bromoethanol with sodium iodide in acetone at room temperature.

Methyl 3-hydroxy-4-nitroso-2-naphthoate was prepared by adding 1 ml concentrate sulfuric acid to a solution of 1.88 grams 2-hydroxy-3-carboxynaphthalene in 100 ml methanol, and refluxing the mixture for 6 hours, to obtain methyl 2-hydroxynaphthoate. The methyl 2-hydroxynaphthoate was filtered and dried under a vacuum. 1.01 gram methyl 2-hydroxynaphthoate was dissolved in 10 ml of an aqueous solution of 1 N NaHCO₃, and 0.68 gram NaNO₂ was added. The mixture was cooled to a temperature of 5-8° C. and then added dropwise to a cooled solution of 6 ml concentrated sulfuric acid in 100 ml water. After 5 hours, the product was filtered, washed twice with water and dried under a vacuum.

Methyl 6-hydroxy-5-nitroso-2-naphthoate was prepared according to the same procedure described above for methyl 3-hydroxy-4-nitroso-2-naphthoate, except that 2-hydroxy-6-carboxynaphthalene was used as a starting material instead of 2-hydroxy-3-carboxynaphthalene.

2-aminoethyl-di(2-hydroxyethyl)amine was prepared by reacting N-tert-butyloxycarbonyl-ethylenediamine (Boc-ethylenediamine) with 2-bromoethanol to obtain Boc-2-aminoethyl-di(2-hydroxyethyl)amine, followed by removal of the Boc protecting group by trifluoroacetic acid in dichloromethane.

O-methacryloyl-N-hydroxysuccinimide was prepared by esterifying methacrylic acid with N-hydroxysuccinimide, using bis-(trichloromethyl) carbonate (BTC) as a coupling agent.

All other chemicals were obtained from Sigma-Aldrich.

### Methods:

Preparation of Polymers:

The polymers were synthesized following the general imprinting protocol previously described by Sellergren [Chem. Mater., 1998, 10:4037-4046].

58.8 mg (˜300 μmol) EDMA (ethylene dimethacrylate) and a lesser amount (typically 0-60 μmol) of one or more functional monomer were dissolved in 500 μl ethyl acetate in a 1.8 clear screw neck vial. For preparing smart polymers, a smart functional monomer was used.

For preparing imprinted polymers (MIPs), a template (15 μmol Erg-TAD-Py, except where stated otherwise) was added, and the solution was gently heated. Thereafter, 0.5 mg of the initiator 2,2′-azobisisobutyrylnitrile (AIBN) was added. For preparing non-imprinted polymers (NIPs), a template was not added.

The reaction mixture was cooled on ice, degassed with bubbling nitrogen gas for 5 minutes and then sealed. Polymerization was accomplished by keeping the solution at a temperature of 70° C. for 20 hours. The resulting polymer was washed in cycles with ethanol followed by intensive centrifugation and separation and then dried at 70° C. overnight.

Uptake Measurements:

5 mg of a tested polymer were washed several times with ethanol. 335 μl of a solution of a target compound (0.05 mg/ml Erg-TAD-Py in ethanol, except where stated otherwise) was added, and the mixture was shaken for 20 minutes (except where stated otherwise) at room temperature. The supernatant was then separated from the polymer and injected into a high performance liquid chromatography (HPLC) apparatus to determine target concentration, as described below. The percentage of the unbound target was calculated from the area of the HPLC peak obtained for the supernatant, by comparison to the peak area obtained for a control sample consisting of the target solution without a polymer. Uptake was expressed as the percentage of target bound to the polymer.

High Performance Liquid Chromatography (HPLC):

A Dionex HPLC apparatus was used for measuring concentrations in solution.

20 μl samples were measured at 23° C. using a flow rate of 1 ml per minute, a mobile phase comprising methanol and water, and a column of 5 μm C-18 (Phenomenex, Inc.).

Calculation of Partition Coefficients and Separation Factors:

To quantify the selectivity of the different MIPs for a given number of targets it is convenient to calculate the corresponding separation factors (α in chromatographic separations) between two given compounds. The MIP binding experiments were interpreted using the batch method described by Spivak et al. [Advanced Drug Delivery Reviews 57 (2005) 1779-1794] for the MIP analysis of binding and selectivity.

After an amount of polymer was mixed with a solution of substrate (S), the amount of substrate remaining free in solution after adsorption to the polymer was measured and referred to as C_{f}. The supernatant was then removed after centrifugation of the mixture, leaving a dried polymer.

The amount of substrate bound (S_{b}) to the MIP was then calculated by subtraction of C_{f} from the total substrate added (Cₜ). Because the polymer is a solid, the amount of bound substrate is divided by the weight of polymer (m) to give the amount of bound substrate per gram of polymer.

S_{b}=[Cₜ−C_{f}]/m

The partition coefficient (Kp) was determined as follows:

Kp=S_{b}/C_{f}

The selectivity of one substrate versus another substrate (separation factor α) was calculated as the ratio of the partition coefficients Kp1 and Kp2 obtained for substrate 1 and substrate 2, respectively.

α=Kp2/Kp1

Thus, the separation factor indicates how many times better substrate 2 binds to a given polymer versus substrate 1.

### Example 1

### Effectiveness of Different Functional Monomers in the Preparation of MIPs for Binding Ergosterol-Triazolinedione-Pyrene (Erg-TAD-Py)

The effectiveness of different functional monomers in the preparation of MIPs for binding an ergosterol-triazolinedione-pyrene (Erg-TAD-Py) adduct was studied by determining the properties MIPs and non-imprinting polymers (NIPs) prepared from various monomer mixtures.

MIPs and NIPs were prepared according to the procedures described hereinabove in the Methods section. For functional monomers, 5.2 mg (60 μmol) MAA (methacrylic acid) was used, as well as an additional 30 μmol of a special functional monomer.

MIP-1 and NIP-1 were prepared using ergosterol methacrylate as the special functional monomer. MIP-2 and NIP-2 were prepared using an equimolar mixture (i.e., 15 μmol of each) of ergosterol methacrylate and 2-(2,4-dinitrophenylamino)ethyl methacrylate as special functional monomers. MIP-3 and NIP-3 were prepared using 2-(2,4-dinitrophenylamino)ethyl methacrylate as the special functional monomer.

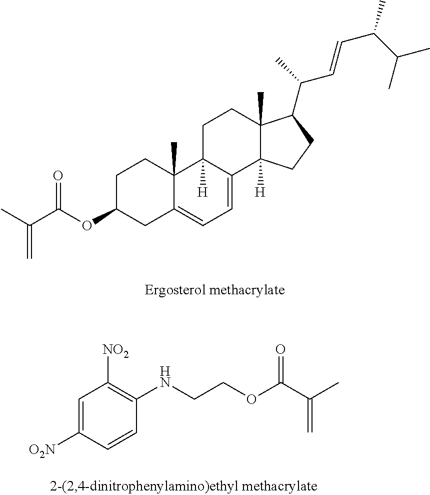

In addition, a poly(ethyleneglycol dimethacrylate) (PEDMA) control NIP was prepared by not including a special functional monomer.

Uptake of Erg-TAD-Py by the abovementioned polymers was then determined as a function of time in accordance with the procedures described hereinabove in the Methods section, with different time periods used for incubation of the polymer and Erg-TAD-Py. The results are shown in Table 1 below and in FIGS. 3A-3C.

TABLE 1
Uptake of Erg-TAD-Py by MIPs and NIPs over time

| | Uptake (wt. %) | | | | | | |
|---|---|---|---|---|---|---|---|
| Time | MIP- | NIP- | MIP- | | | | |
| (minutes) | 1 | 1 | 2 | NIP-2 | MIP-3 | NIP-3 | PEDMA |
| 0 | 0 | 0 | 0 | 0 | 0 | 0 | 0 |
| 7 | 18.81 | 13.73 | 14.44 | 9.64 | 6.66 | 4.04 | 5.18 |
| 15 | 22.28 | 16.77 | 15.22 | 12.05 | 7.69 | 4.74 | 5.87 |
| 30 | 19.39 | 16.16 | 14.49 | 11.48 | 4.99 | 5.87 | 6.11 |
| 60 | 23.00 | 17.54 | 16.94 | 15.73 | 6.55 | 11.98 | 6.74 |
| 120 | 20.09 | 15.52 | 14.45 | 11.84 | 6.93 | 5.94 | 5.82 |

As shown in Table 1 and in FIGS. 3A-3C, saturation of all of the polymers was reached within about 15 minutes.

As further shown therein, polymers prepared using ergosterol methacrylate as the special functional monomer (i.e., MIP-1, NIP-1) exhibited the highest uptake, polymers prepared using a dinitrophenyl (DNP) derivative (i.e., 2-(2,4-dinitrophenylamino)ethyl methacrylate) as the special functional monomer (i.e., MIP-3, NIP-3) or with no special functional monomer (i.e., PEDMA) exhibited the lowest uptake, and polymers prepared using a mixture of ergosterol methacrylate and DNP derivative as the special functional monomer (i.e., MIP-2, NIP-2) exhibited an intermediate uptake. These results suggest that inclusion of an ergosterol-containing monomer increases affinity of a polymer to Erg-TAD-Py, whereas inclusion of a DNP-containing monomer does not improve affinity.

As shown in Table 1 and FIGS. 3A and 3B, MIPs prepared using ergosterol methacrylate as the special functional monomer (i.e., MIP-1 and MIP-2) exhibited significantly higher affinity to the Erg-TAD-Py target than did the corresponding NIPs (i.e., NIP-1 and NIP-2). These results indicate that the desired molecular imprinting was present in the MIPs.

The binding of the Erg-TAD-Py target by the polymers was further studied by comparing binding of Erg-TAD-Py to binding of ergosterol and a pyrene ester derivative (ethyl 4-(pyren-1-yl)butanoate), which represent different moieties present in Erg-TAD-Py.

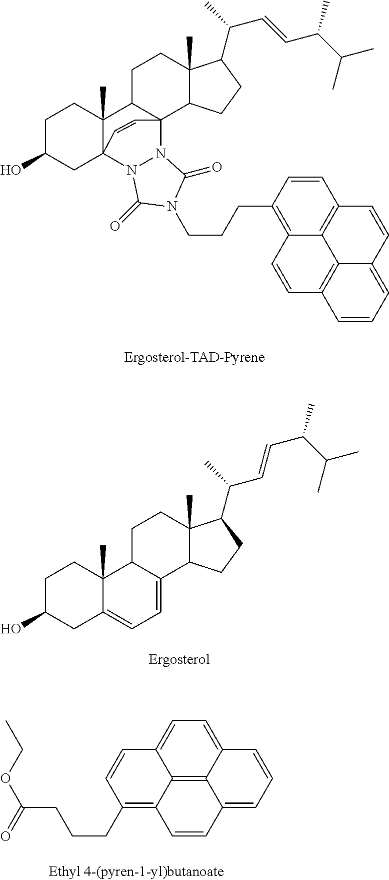

Uptake of each of the three compounds was measured independently as described hereinabove. In addition, a competitive experiment was performed wherein binding of each of the compounds was determined in the presence of an equimolar mixture of the three compounds. Uptake was measured 15 minutes after mixing the polymer and target. The results are shown in Table 2 below.

TABLE 2
Uptake of Erg-TAD-Py, pyrenyl ester and ergosterol by MIPs and NIPs
Selective uptake (wt. %)

| | Separate Targets | | | Mixture of Targets | | |
|---|---|---|---|---|---|---|
| | Pyrenyl | Ergos- | Erg- | Pyrenyl | Ergos- | Erg- |
| Polymer | ester | terol | TAD-Py | ester | terol | TAD-Py |
| MIP-1 | 10.02 | 2.00 | 22.28 | 8.75 | 1.86 | 10.64 |
| NIP-1 | 7.91 | 1.84 | 16.77 | 8.33 | 1.78 | 8.88 |
| MIP-2 | 8.89 | 1.83 | 15.22 | 9.21 | 1.20 | 10.94 |
| NIP-2 | 5.23 | 0.84 | 12.05 | 5.77 | 0.45 | 6.50 |
| MIP-3 | 6.12 | −0.16 | 7.69 | 5.86 | 0.28 | 3.77 |
| NIP-3 | 2.66 | 0.42 | 4.74 | 4.26 | 0.42 | 2.49 |
| PEDMA | 4.77 | 0.58 | 5.87 | 5.67 | 0.27 | 2.94 |

In addition, the selectivity of the MIPs to each of the three targets is expressed as partition coefficients (Kp) in FIG. 4. The selectivity of the MIPs towards a particular target is expressed as separation factors (α) in Table 3 below.

TABLE 3
Separation factors of MIP-1 and MIP-2

| | | Separation factor (α) | |
|---|---|---|---|
| | | K_{P (Adduct)}/ | K_{P (Adduct)}/ |
| | Polymer | K_{P (Pyrenyl)} | K_{P (Ergosterol)} |
| Targets measured separately | MIP-1 | 2.5 | 13.4 |
| | MIP-2 | 2.3 | 9.4 |
| Targets measured in mixture | MIP-1 | 1.2 | 6.4 |
| | MIP-2 | 1.1 | 13.3 |

As shown in Tables 2 and 3 and in FIG. 4, the polymers bind to the pyrenyl ester more strongly than to ergosterol, and to the Erg-TAD-Py adduct more strongly than to either pyrenyl ester or ergosterol. These results indicate that the ergosterol moiety of the Erg-TAD-Py adduct seems to have minimal contribution to molecular recognition, whereas the pyrenyl moiety plays a greater role, but that the whole adduct is required to achieve maximal affinity.

In addition, the results shown in Table 2 confirm that inclusion of ergosterol-containing monomers increases the affinity of the polymers to Erg-TAD-Py, and that MIPs exhibit a higher affinity than NIPs.
In view of the above results, MIP-1, which exhibited the strongest affinity for Erg-TAD-Py, was selected as a basis for experiments with smart MIPs.

### Example 2

### Molecular-Imprinted Smart Polymers (MISPs)

MIPs and NIPs were prepared by polymerizing EDMA, MAA and ergosterol methacrylate, as described in Example 1 for MIP-1 and NIP-1. To produce a molecular-imprinted smart polymer (MISP) and a non-imprinted smart polymer (NISP), 23 μmol of a smart functional monomer was polymerized along with the EDMA, MAA and ergosterol methacrylate.

Rhodamine B is a halochrome molecule which becomes fluorescent as a result of a drop in pH to below approximately pH 4.

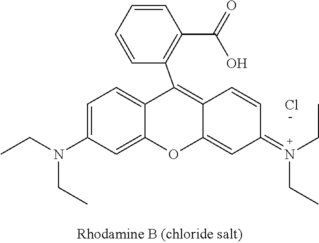

Acceptance of a hydrogen ion results in generation of a fluorescent species, which is accompanied by a large conformational change and creation of a local positive charge. In the present Example, an amide derivative of rhodamine B was used, in which the carboxylic acid group of rhodamine B is replaced by an amide group. The conformational change of such derivatives as a result of acceptance of a hydrogen ion is as follows:

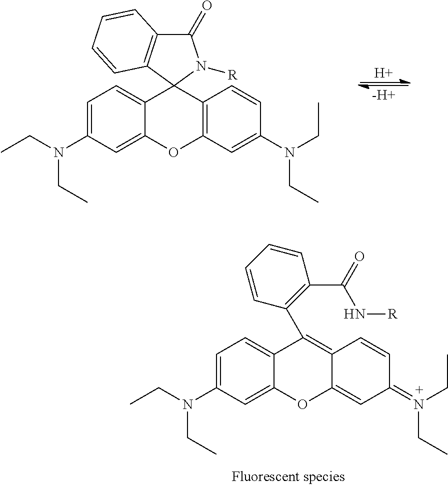

Smart monomers derived from rhodamine B amide derivatives, which take advantage of the change in charge and conformation that the molecule undergoes following a drop in pH, were prepared, having the structure:

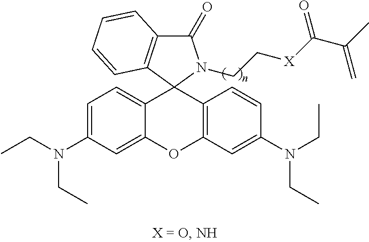

wherein n is an integer from 1 to 5.

The above Rhodamine B amide derivatives were prepared according to the procedures depicted in Scheme 1.

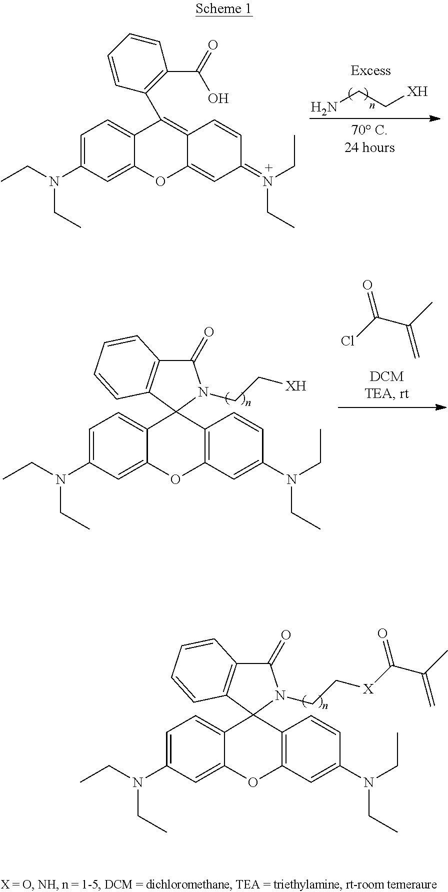

The benzospiropyran scaffold has been applied for generation of photocromic molecules, polymers and materials (e.g., as photochromic coating for lenses) characterized by the following reversible transition:

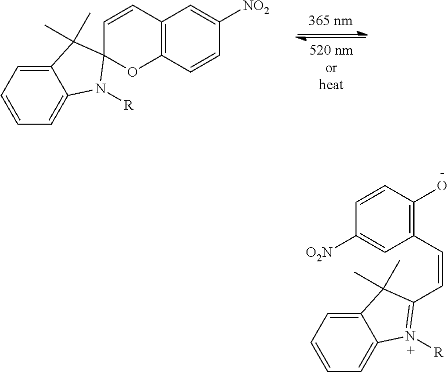

The following benzospiropyran derivative was prepared and used as a smart monomer:

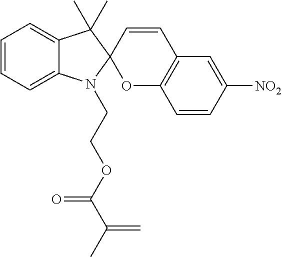

The preparation of the above smart monomer is depicted in Scheme 2.

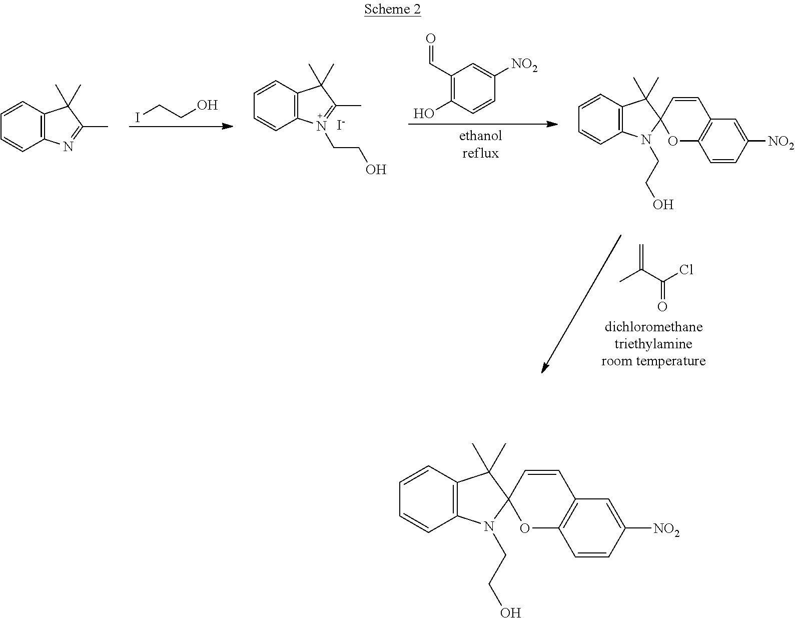

Fluorescein is a halochromic molecule which is very sensitive to pH changes and equilibrates between several ionization states. In aqueous solutions, the neutral form presents an equilibrium between the closed lactone and the open protonated acid, whereas a characteristic open di-anion form is attained at basic conditions (pH>8). It becomes fluorescent at neutral and basic pH environment, from pH 6.5 to pH 9. The generation of the fluorescent species triggered by a pH change is accompanied by a considerable conformational change and by the generation of a local negative charge:

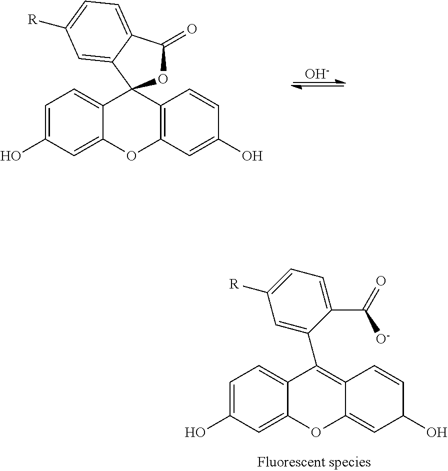

Smart monomers derived from fluorescein were prepared having the structure:

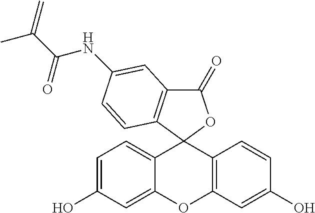

The above fluorescein derivative was initially prepared by protecting the hydroxy groups of aminofluorescein with pivaloyl protecting group, reacting the amine group with methacryloyl chloride, and removing the pivaloyl groups, as depicted in Scheme 3.

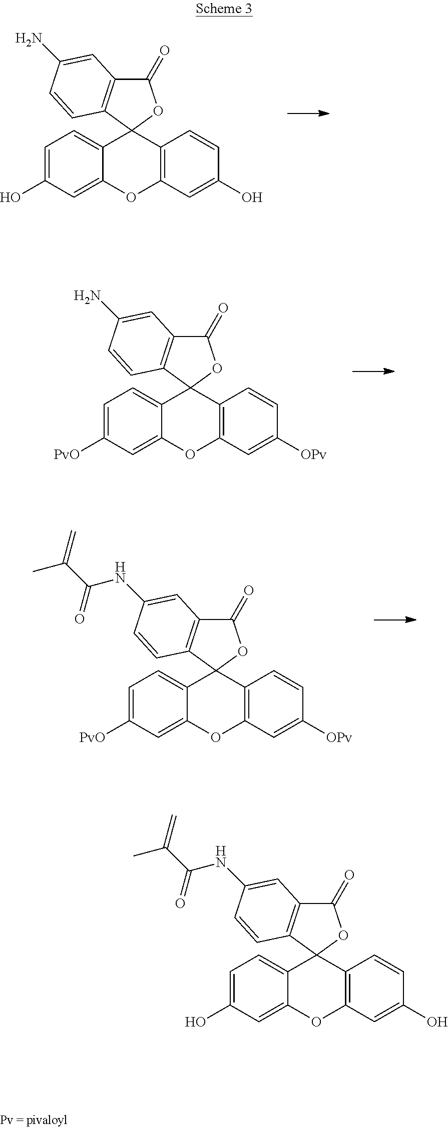

NISPs were prepared using the above three smart monomers.

In addition, MISPs were prepared using the above Rhodamine-B-based smart monomer (wherein n=1 and X=oxygen) and benzospiropyran-based smart monomer.

As shown in FIG. 5, all three NISPs underwent reversible and recursive cycles of activation and deactivation, as judged by the color change of the samples.

Rhodamine B-derived smart polymers were activated by being exposed to 10% trifluoroacetic acid in ethanol, followed by washing with ethanol.

Benzospiropyran-containing smart polymers were activated by UV irradiation (˜360 nm) for 20 minutes.

Fluorescein-containing smart polymers were activated by exposure to 1% NaOH in ethanol, followed by washing with ethanol.

Uptake of Erg-TAD-Py (0.05 mg/ml in ethanol) by the MISPs and corresponding NISPs was determined, and the results are shown in Table 4 below. Uptake was measured both before and after activation of the smart polymers. The corresponding MIP and NIP served as a control.

TABLE 4
Uptake of Erg-TAD-Py by smart polymers
before and after activation

Uptake %

| | | Before | After |
|---|---|---|---|
| Smart monomer type | Polymer | activation | activation |
| Rhodamine B | MISP-1 | 23.01 | 20.18 |
| | NISP-1 | 21.46 | 19.82 |
| Benzospiropyran | MISP-2 | 21.28 | 17.44 |
| | NISP-2 | 19.18 | 16.07 |

| | | |
|---|---|---|
| none | Control MIP | 22.5 |
| | Control NIP | 16.7 |

As shown in Table 4, Rhodamine B derivatives increased affinity of the polymers to Erg-TAD-Py over that of the corresponding control polymers, and benzospiropyran derivatives had little if any effect on the affinity. Notably, activation of both smart polymers resulted in decreased affinity to the Erg-TAD-Py target.

As shown in FIG. 6A, the Rhodamine B-derived MISP exhibits higher uptake than does the corresponding Rhodamine B-derived NISP, indicating the presence of an imprinting effect, and activation of the smart polymer decreases uptake more effectively in the MISP than in the NISP.

Similarly, as shown in FIG. 6B, the benzospiropyran-containing MISP exhibits higher uptake than does the corresponding benzospiropyran B-containing NISP, indicating the presence of an imprinting effect, and activation of the smart polymer decreases uptake more effectively in the MISP than in the NISP.

These results indicate that the effect of the Rhodamine B-based and benzospiropyran-based smart monomers is accentuated in the specific binding sites of the imprinted polymers relative to non-specific binding sites.

In order to determine the reusability of the smart polymers, the benzospiropyran-containing polymers (i.e., MISP-2 and NISP-2) were exposed to successive cycles of activation and deactivation, and uptake was determined at each stage.

First MISP-2 and NISP-2 polymers were converted to the deactivated merocyanine form of the benzospiropyran by gently warming the polymers at a temperature of 50° C. in the dark. Uptake of Erg-TAD-Py by the deactivated polymers were then determined as described in the Methods section. Uptake of the activated polymer was then determined by measuring uptake after incubation of the polymers under UV light (˜360 nm) for 20 minutes with a new solution of Erg-TAD-Py. Subsequent cycles repeating the above activation and deactivation procedures followed.

As shown in FIG. 7A, after repeated cycles, a decrease in uptake by the MISP and NISP smart polymers was noticeable.

In contrast, as shown in FIG. 7B, specific uptake, calculated as the difference in uptake between the MISP and the corresponding NISP, did not exhibit a decreasing trend. As further shown in FIG. 7B, specific uptake decreases significantly following activation of the smart polymer. These results indicate that the specific binding resulting from molecular imprinting is reduced by activation of the MISP, and is fully recovered by deactivation of MISP.

### Example 3

### Effect of MISP Activation on Release of Erg-TAD-Py Template

A benzospiropyran-containing MISP was prepared as described for MISP-2 in Example 2. The polymer was then divided into two equal portions, which were thoroughly washed and then exposed to a solution of 0.05 mg/ml Erg-TAD-Py in ethanol. After an incubation period of 20 minutes, the supernatant was removed from each sample and a solution of 0.01 mg/ml Erg-TAD-Py was added. This dilute solution was used as a washing medium. One portion of the MISP was kept in the deactivated merocyanine form, while the second portion was converted by exposure to UV irradiation to the activated zwitterionic form, as described in Example 2.

The addition of the dilute solution of Erg-TAD-Py was intended to avoid the total release of the template from the polymer, as washing with pure ethanol might result in full release from both forms of the polymers, thereby making it more difficult to distinguish between the two forms of the polymer.

The amount of Erg-TAD-Py template remaining in the polymer before washing was determined by HPLC as described in the Methods section for uptake measurements, and considered as 100%. Following a single washing using the dilute solution of Erg-TAD-Py in ethanol, the percentage of Erg-TAD-Py which was released was calculated by subtracting the amount of Erg-TAD-Py originally in the washing solution (0.01 mg/ml) from the final amount of Erg-TAD-Py in the supernatant as determined by HPLC.

As shown in FIG. 8, activation of the MISP resulted in more effective release of the Erg-TAD-Py template from the polymer.

This result indicates that smart monomers can be used to enhance release of templates from MIPs.

### Example 4

### Effectiveness of Different Polymers in the Preparation of MIPs for Binding Ergosterol-Triazolinedione-Pyrene

In order to improve the properties of MIPs which bind Erg-TAD-Py, various parameters of the synthesis of MIPs described in Example 1 were altered.

As the methacrylic acid (MAA) used may be too polar to optimally bind the relatively hydrophobic Erg-TAD-Py, MIPs and NIPs were prepared according to the procedures described above in the Methods section, using 60 μmol methyl methacrylate

(MMA) as the functional monomer, instead of the more polar MAA used in Example 1, and 300 μmol NOBE (N,O-bismethacryloyl ethanolamine) instead of EDMA, in order to facilitate hydrogen bonding. For comparison, MIPs and NIPs were also prepared from EDMA with 60 μmol MAA as the functional monomer. In addition, MIPs and NIPs were prepared from EDMA and NOBE without MAA or MMA (i.e., without a functional monomer).

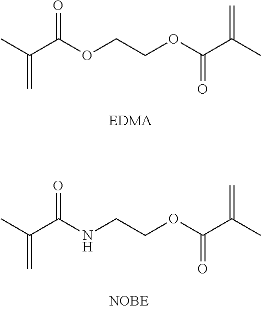

Uptake of Erg-TAD-Py was determined for each of the polymers following 20 minutes incubation with Erg-TAD-Py. The effect of cycles of acidic and basic conditions on uptake was also determined.

As shown in FIG. 9A, the NOBE-MMA MIP exhibited higher uptake to the target than did the corresponding NIP throughout 2 cycles of acidic/basic conditions, indicating conservation of the imprinting effect, whereas the EDMA-MAA MIP exhibited little, if any, imprinting effect after exposure to acidic and basic conditions. In addition, the uptake of the EDMA-MAA polymers was pH dependent.

As further shown in FIG. 9A, the EDMA-MAA polymers exhibited a higher affinity to Erg-TAD-Py than did the NOBE-MMA polymers.

Similarly, as shown in FIG. 9B, the EDMA polymers exhibited a higher affinity to Erg-TAD-Py than did the NOBE polymers. However, as further shown in FIG. 9B, the EDMA and NOBE MIPs without MAA or MMA exhibited a significantly larger imprinting effect than did the MIPs with MAA or MMA. Moreover, the binding by the polymers was not pH-dependent.

NOBE and EDMA have very similar structures, but NOBE is more hydrophilic. As the affinity exhibited by EDMA polymers is considerably greater than the affinity exhibited by NOBE polymers, these results suggest that polarity of the polymer matrix plays a considerably role in binding to the Erg-TAD-Py adduct.

To test this hypothesis MIPs and NIPs were prepared from both NOBE and EDMA, using 60 μmol bisphenol A dimethacrylate as the functional monomer in order to add aromatic groups to the polymers. Uptake was then measured as described above.

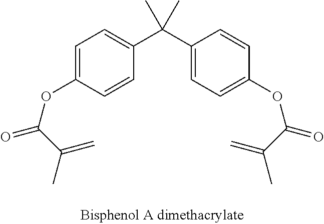

As shown in FIG. 10, the affinity of EDMA and NOBE polymers with bisphenol A was approximately twice that of the corresponding polymers without bisphenol A. However, the EDMA-based MIP with bisphenol A exhibited relatively small imprinting effect.

These results suggest that reduced hydrophilicity and/or π-π interactions (e.g., between the pyrene moiety of Erg-TAD-Py and the bisphenol A) increases affinity of polymers to Erg-TAD-Py.

In addition, EDMA-based MIPs without MAA appear to provide the best combination of affinity and molecular imprinting.

The above results indicate that the affinity of MIPs and MISPs to a target can be modulated by selecting appropriate monomers.

### Example 5

### Effect of Solvent on Binding of Erg-TAD-Py by MIPs

All binding Experiments in the above Examples were performed using ethanol, a protic, polar solvent with a dielectric constant of 24.3, as solvent. Ethanol was found to be suitable for dissolving Erg-TAD-Py target at a concentration of 0.05 mg/ml (about 60 uM).

The uptake of Erg-TAD-Py by the EDMA-based MIP and NIP described in Example 4 (without MAA or bisphenol A) was determined as described hereinabove using solutions of 0.05 and 0.01 mg/ml Erg-TAD-Py in ethanol.

As shown in FIG. 11, there was no significant difference in uptake between the two concentrations of Erg-TAD-Py. This result indicated that both concentrations are above the binding constant range and that further studies can be performed using only 0.01 mg/ml Erg-TAD-Py. This in turn suggested that measurements can be performed in serum, which is particularly useful for performing biologically relevant measurements.

The solubility of Erg-TAD-Py in mixtures of 10%, 30% and 50% DMSO (dimethyl sulfoxide) in water or inactivated fetal calf serum was determined by measuring the concentration of dissolved Erg-TAD-Py using HPLC, and the results are summarized in Table 5 below.

TABLE 5
Solubility of Erg-TAD-Py in mixtures of DMSO in water or serum
DMSO in water

| | | | 0.1 mg/ml | Concentration |
|---|---|---|---|---|
| | | | Erg-TAD-Py | of dissolved |
| | H₂O | DMSO | in DMSO | Erg-TAD-Py |
| % DMSO | (μl) | (μl) | (μl) | (mg/ml) |
| 10 | 900 | — | 100 | 0.0007 |
| 30 | 700 | 200 | 100 | 0.0067 |
| 50 | 500 | 400 | 100 | 0.0060 |

DMSO in serum

| | | | 0.1 mg/ml | Concentration |
|---|---|---|---|---|
| | | | Erg-TAD-Py | of dissolved |
| | Serum | DMSO | in DMSO | Erg-TAD-Py |
| % DMSO | (μl) | (μl) | (μl) | (mg/ml) |
| 10 | 900 | — | 100 | 0.0078 |
| 30 | 700 | 200 | 100 | 0.0084 |
| 50 | 500 | 400 | 100 | 0.0068 |

As shown in Table 5, 30% DMSO in either water or serum afforded the highest solubility of Erg-TAD-Py. Such a solvent is advantageous in that it will not cause denaturation of serum proteins.

The measurement of the uptake of Erg-TAD-Py by the abovementioned EDMA-based MIP and NIP was therefore repeated using a solution of 0.01 mg/ml Erg-TAD-Py in serum with 30% DMSO instead of in ethanol.

As shown in FIG. 12, both total and specific binding are considerably greater in serum/DMSO than in ethanol. This result suggests that a polar environment enhances uptake capabilities of the polymers. In addition, serum proteins may enhance specific binding by the MIP by inhibiting non-specific binding, which is a known property of some serum proteins.

The above results indicate that the affinity of MIPs and MISPs to a target can be modulated by selecting a solvent with appropriate properties.

### Example 6

### Indolenine-Based Smart Polymers

Indolenine is of particular interest for use in smart polymers, as indolenine derivatives can reversibly generate a positive charge after being triggered by either light or a pH change. The benzospiropyran-based monomer described in Example 2 is also an indolenine derivative. A further indolenine-based monomer was prepared having the following two forms, an open-ring form and a closed-ring form:

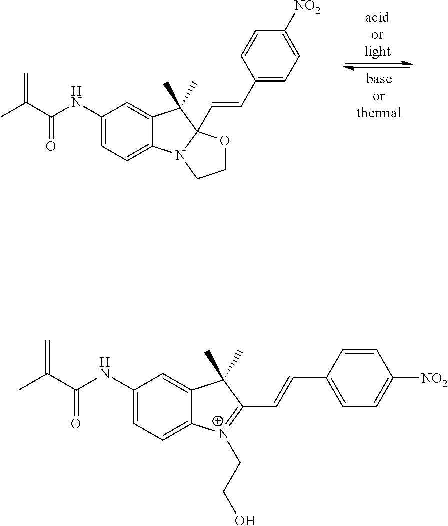

The transition between the two forms of the monomer is characterized by generation or elimination of a full charge with relatively little conformational change.

The monomer was synthesized as depicted in Scheme 4.

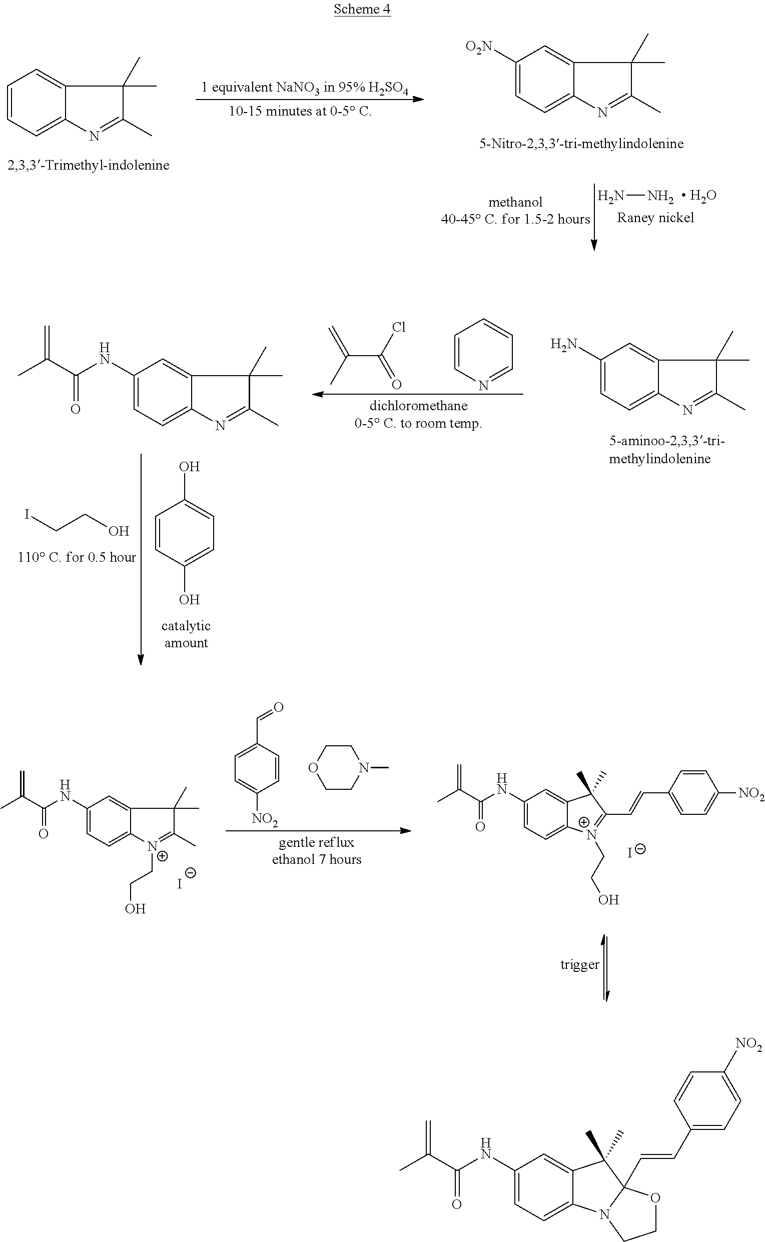

A MISP and NISP were prepared as described in Example 2, using 360 μmol EDMA, and 30 μmol ergosterol methacrylate and 23 μmol of the above indolenine-based smart monomer as functional monomers. As a control, a MIP and NIP were prepared using 30 μmol ergosterol methacrylate as functional monomer without a smart monomer. The polymers were washed several times with ethanol.

The polymers were then tested for uptake of Erg-TAD-Py both before and after activation by treatment with trifluoroacetic acid (TFA), as described in Example 2.

As shown in FIG. 13, treatment with TFA reduced uptake by the smart polymers but not uptake by the control polymers, reducing the uptake of the MISP from 34% to 27% and the uptake of the NISP from 31% to 26%. In addition, the MISP exhibited higher uptake than the NISP.

These results indicate that the positive charge generated by activation of the indolenine-based monomer affects uptake, and that the MISP exhibits selectivity due to imprinting. The results therefore corroborate the hypothesis that electrostatic factors play an important role in the binding constant and can be used to afford a desired effect following a suitable signal.

### Example 7

### MISPs Prepared from Smart Monomers Comprising an Attached Target Molecule

MISPs were designed so as to have one smart monomer residue per binding site. To this effect, “third generation” smart monomers derived from Rhodamine B were synthesized. Third generation smart monomers are characterized by the inclusion of a moiety which corresponds to the target molecule, the moiety being attached to the rest of the molecule by a labile bond.

The target molecule selected in the preparation of the MISPs was 9-fluorenyl methanol (9FM).

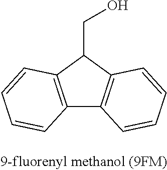

The smart monomer was of a type referred to herein as “Y-type”, in which a target (9FM) moiety which acts as a template for molecular imprinting, a polymerizable moiety (methacryl) and a responsive moiety (Rhodamine B amide derivative) are each connected to a central scaffold (2-amino-5-hydroxybenzoic acid). For comparison, a monomer was prepared without the 9FM moiety, in order to prepare a non-imprinted smart polymer (NISP).

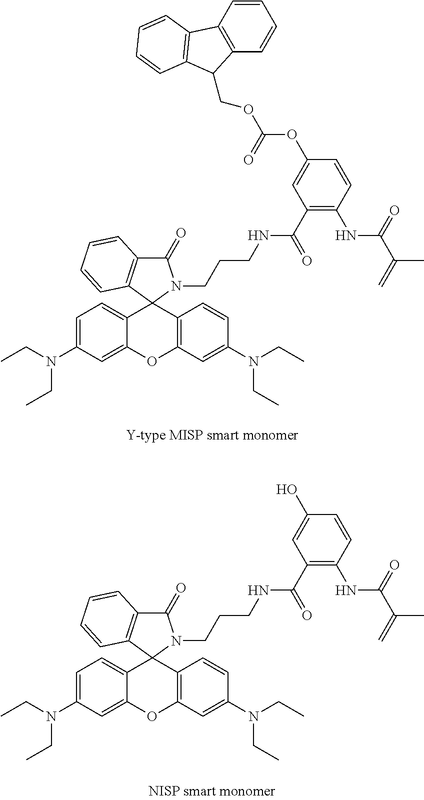

The MISP smart monomer and the NISP smart monomer were prepared as depicted in Scheme 5:

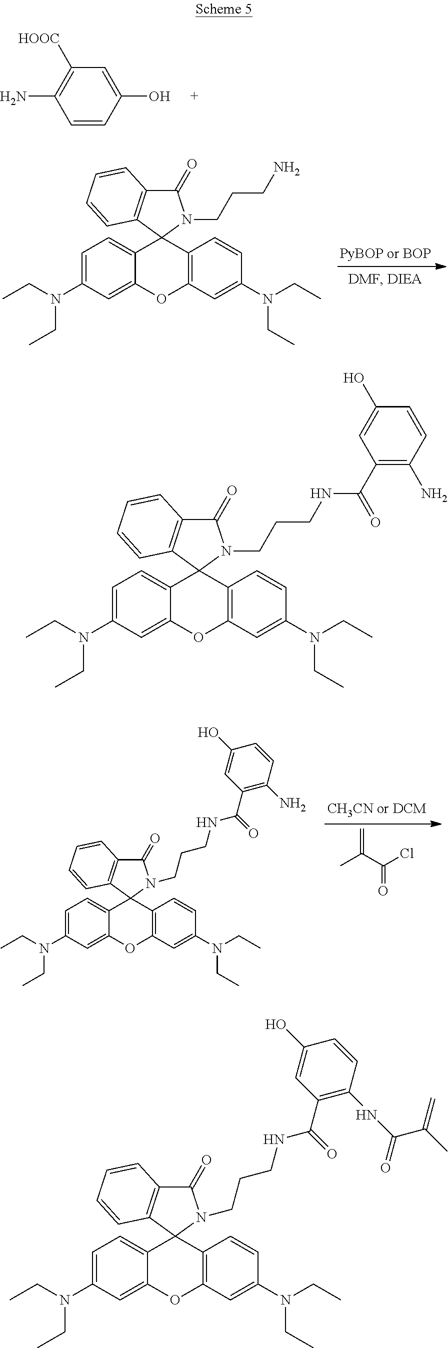

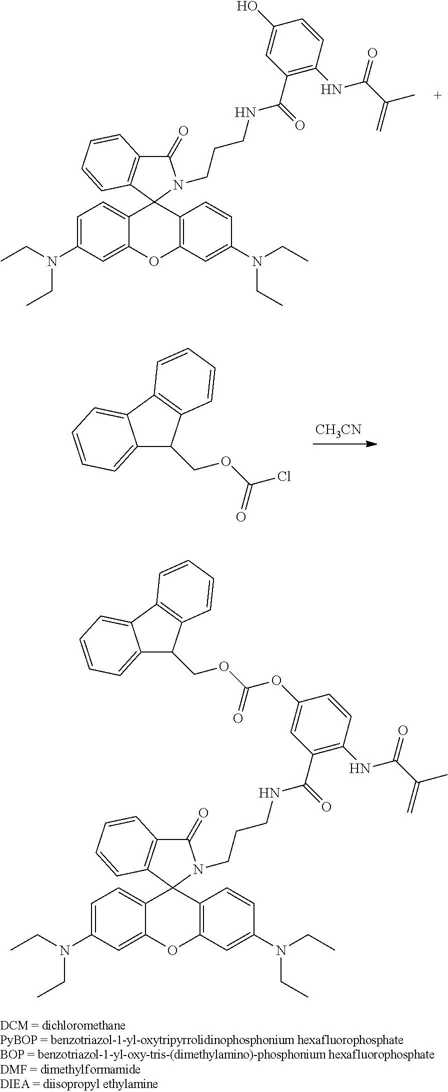

A “third generation” MISP was prepared according to the procedures described in the Methods section, with 36.6 μmol of the above Y-type MISP smart monomer as the functional monomer. After polymerization, 9FM was released from the polymer by treatment with a solution of 25% piperidine.

As a control, a NISP was prepared according to the procedures described in the Methods section, with 36.6 μmol of the above NISP smart monomer as the functional monomer.

Uptake of 9FM by each polymer was determined as described hereinabove using a solution of 0.05 mg/ml 9FM. Uptake by the polymers was measured both before and after activation of the polymers with 1% trifluoroacetic acid.

As shown in FIG. 14, the “third generation” MISP exhibited considerably greater binding of the target than did the control NISP, and that uptake by the MISP was reduced to a large extent following activation of the MISP. In addition, the uptake by the activated MISP is approximately at the level of uptake by the activated NISP.

These results indicate that the “third generation” monomers with a covalently linked target molecule successfully imprinted selective binding pockets for the target (9FM) in the smart polymer, and that the smart polymer releases bound target from the selective binding pockets following activation.

### Example 8

### Additional Smart Monomers Comprising a Polymerizable Moiety

The syntheses of additional smart monomers comprising benzospiropyran, spirooxazine, fluorescein or malachite green moieties and one polymerizable moiety are described below.

A. Benzospiropyran Derivatives

Two new benzospiropyran derivatives were prepared, each having a methacryloyl group to allow polymerization, thereby making the derivative useful as a smart monomer.

The first derivative had the structure:

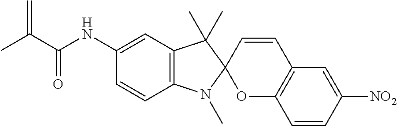

This smart monomer was prepared as depicted in Scheme 6:

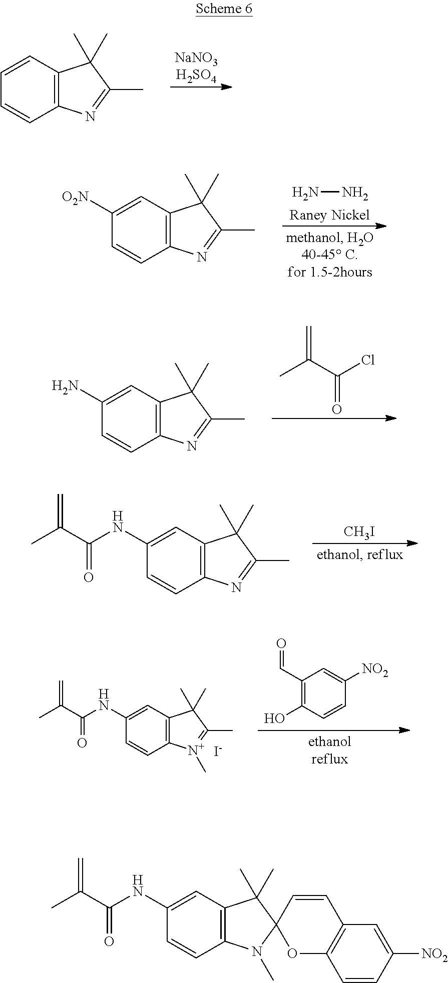

The second derivative had the structure:

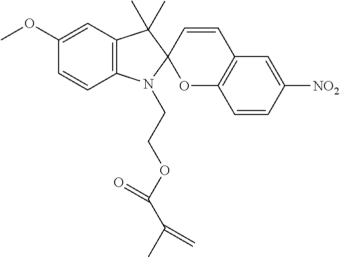

This smart monomer was prepared as depicted in Scheme 7:

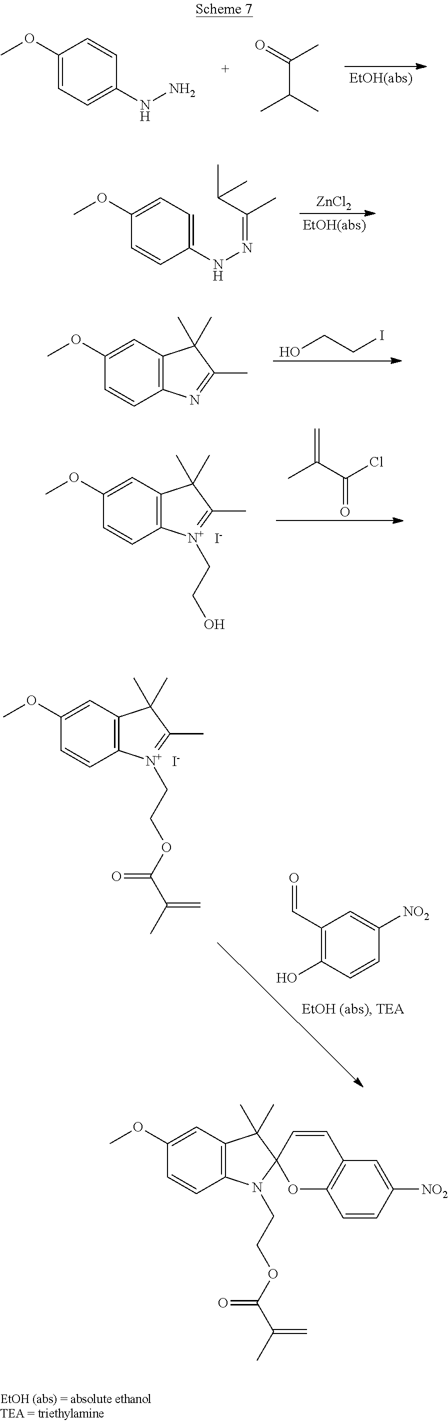

B. Spirooxazine Derivatives

A spirooxazine derivative smart monomer was prepared having the structure:

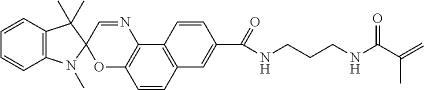

The spirooxazine functionality is closely related to the spiropyran functionality, differing in that the oxazine ring has a nitrogen atom where the pyran ring has a carbon atom.

This smart monomer was prepared from 1,3,3-trimethyl-2-methyleneindoline and methyl 6-hydroxy-5-nitroso-2-naphthoate, as depicted in Scheme 8.

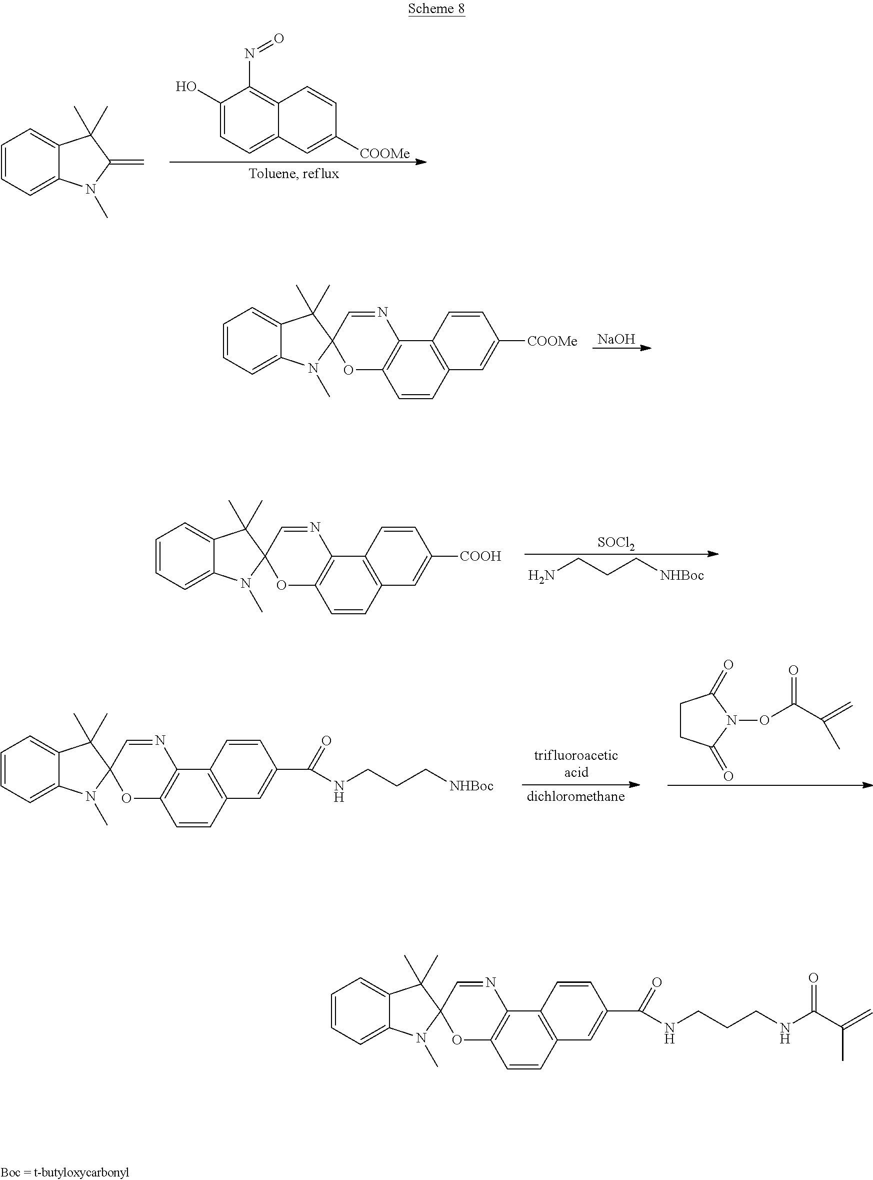

In addition, an alternative intermediate was prepared using methyl 3-hydroxy-4-nitroso-2-naphthoate instead of methyl 6-hydroxy-5-nitroso-2-naphthoate, as depicted in Scheme 9. A smart monomer is then obtained from this intermediate using the procedures described above in Scheme 8.

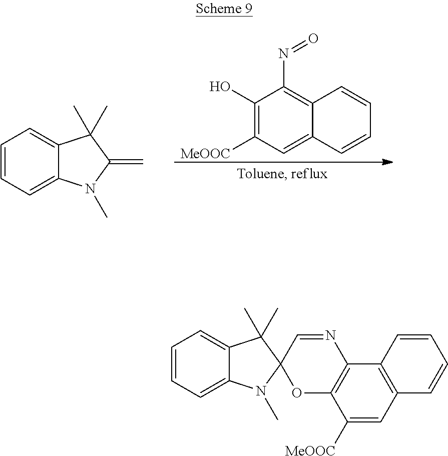

In addition, another spirooxazine derivative was prepared using 1-(2-hydroxyethyl)-3,3-dimethyl-2-methyleneindoline and 1-nitroso-2-hydroxynaphthalene as starting materials, as depicted in Scheme 10. A smart monomer is then obtained from this intermediate by reacting the intermediate with methacryloyl chloride in dichloromethane with triethylamine.

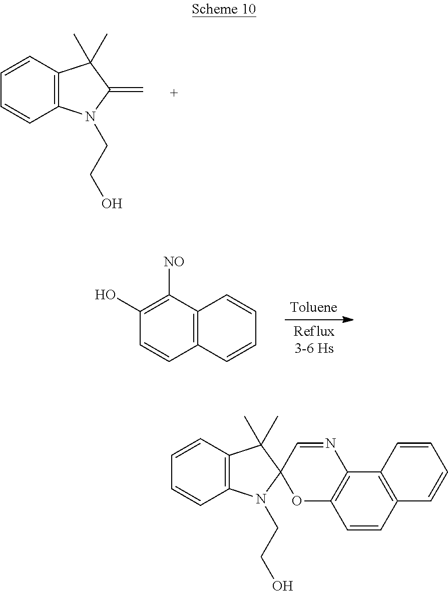

C. Fluorescein Derivatives

A fluorescein derivative was prepared having the structure:

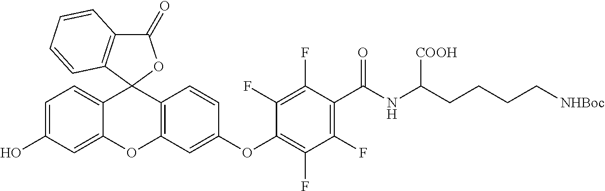

This derivative was prepared from fluorescein and methyl perfluorobenzoate, followed by amidation with N-tert-butyloxycarbonyl-lysine (Boc-Lys) as depicted in Scheme 11. The derivative is then converted to a smart monomer by removing the Boc (t-butyloxycarbonyl) protecting group to obtain a free amine group and reacting the compound with O-methacryloyl-N-hydroxysuccinimide to obtain a monomer with a methacrylamide moiety, as described above for spirooxazine derivatives (e.g., in Scheme 8).

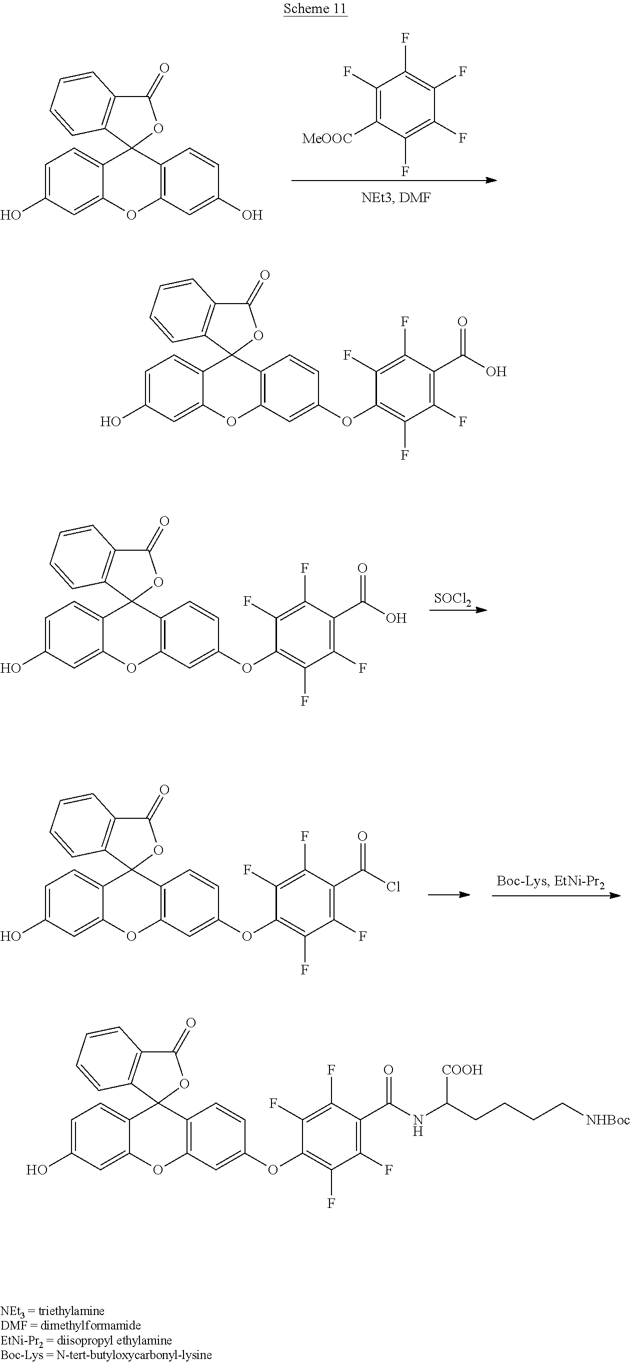

D. Malachite Green Derivatives

A malachite green derivative was prepared having the following structure:

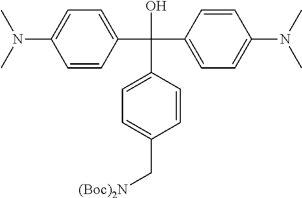

The derivative was prepared by reacting N-tert-butyloxycarbonyl-4-aminomethylbenzoic acid with di-tert-butyl dicarbonate ((Boc)₂O) for two days with triethylamine and 4-dimethylaminopyridine (DMAP) to obtain the following doubly Boc-protected intermediate:

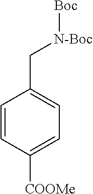

This intermediate was then reacted in dry tetrahydrofuran with a Grignard reagent prepared from magnesium and 4-bromo-N,N-dimethylaniline to obtain the abovementioned derivative.

A smart monomer is prepared from the abovementioned malachite green derivative by removing the Boc groups with an acid (e.g., trifluoroacetic acid) and reacting the free amine with methacryloyl chloride.

The smart monomer is activated by light to generate a positively charged form as follows:

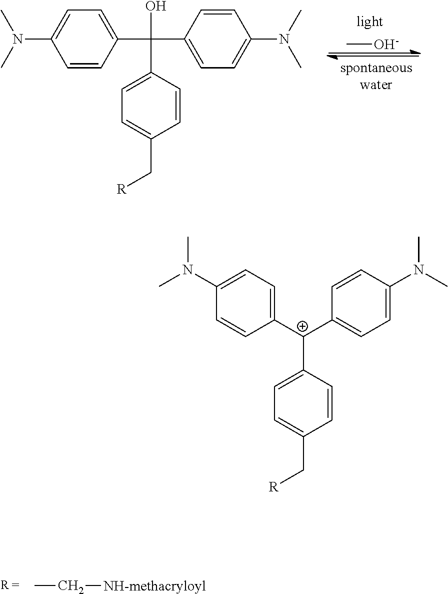

### Example 9

### Additional Smart Monomers Comprising Two Polymerizable Moieties

The syntheses of additional smart monomers having rhodamine B or benzospiropyran moieties and two polymerizable moieties are described below.

A. Rhodamine B Derivatives

A dimethacrylate ester derivative of rhodamine B and corresponding dimethacrylamide derivative were prepared from rhodamine B. The dimethacrylate ester was produced by reacting rhodamine B with 2-aminoethyl-di(2-hydroxyethyl)amine. And the dimethacrylamide was produced by reacting rhodamine B with tri(2-aminoethyl)amine, as shown in Scheme 12.

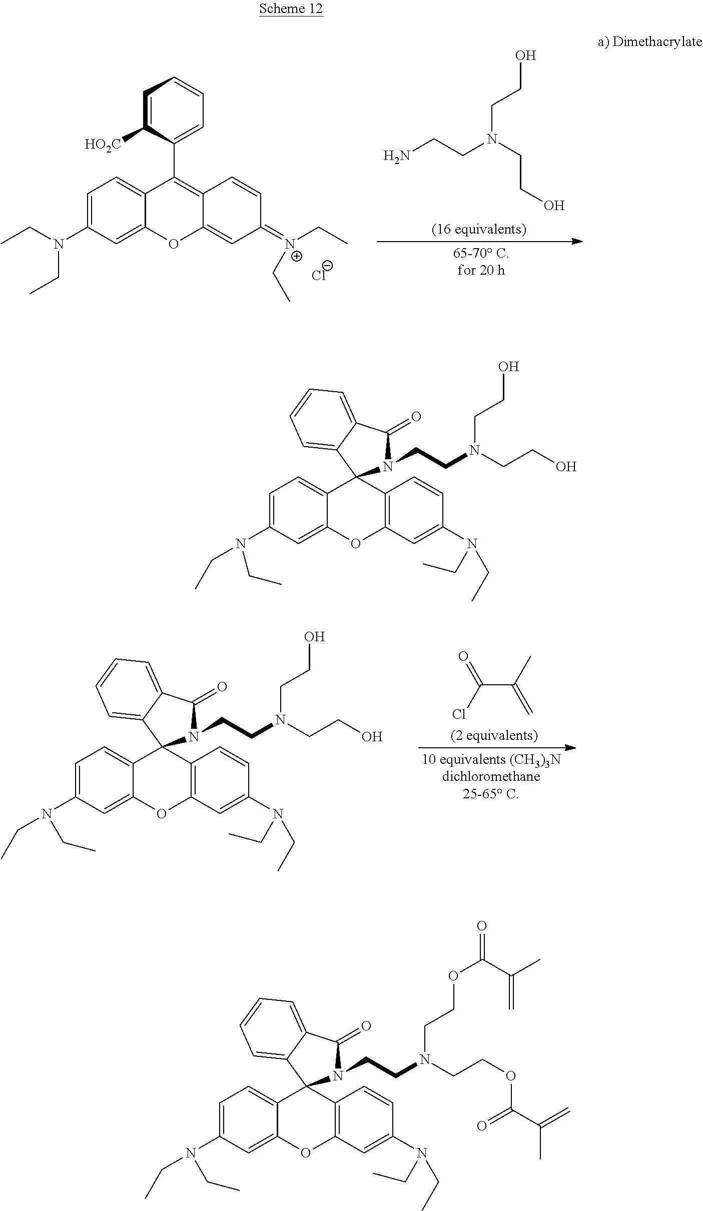

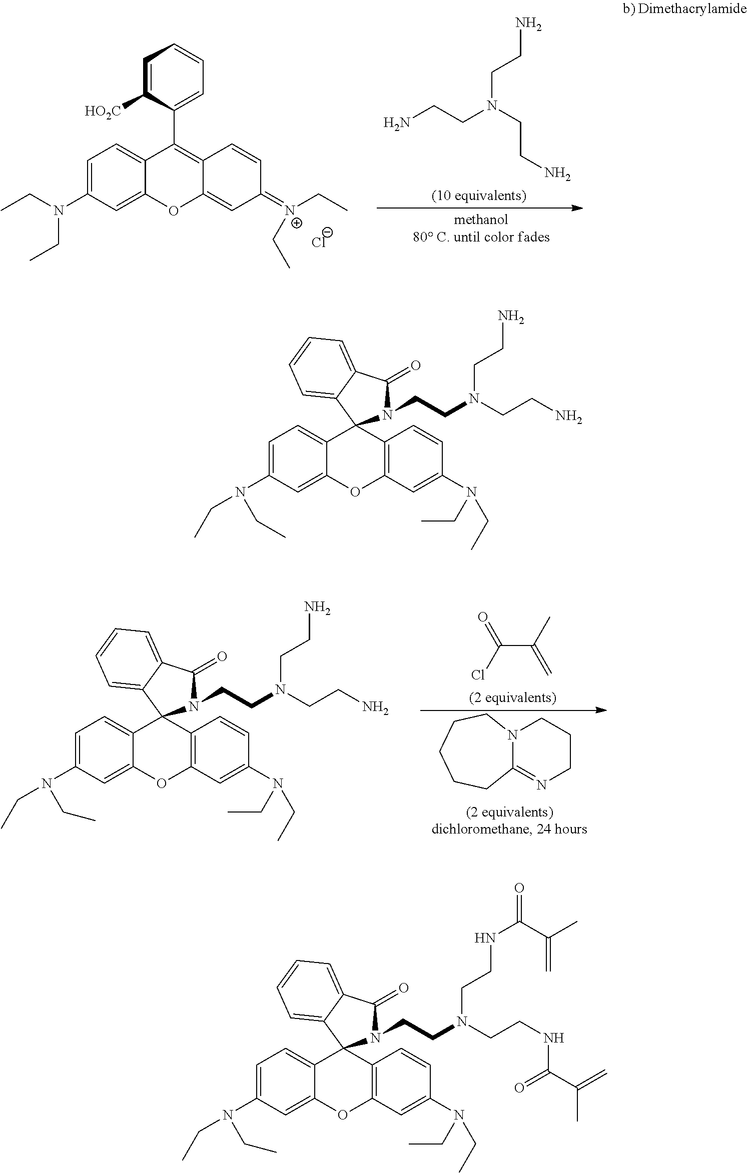

Another dimethacrylamide derivative of rhodamine B, having a lysine linker moiety, was prepared as shown in Scheme 13.

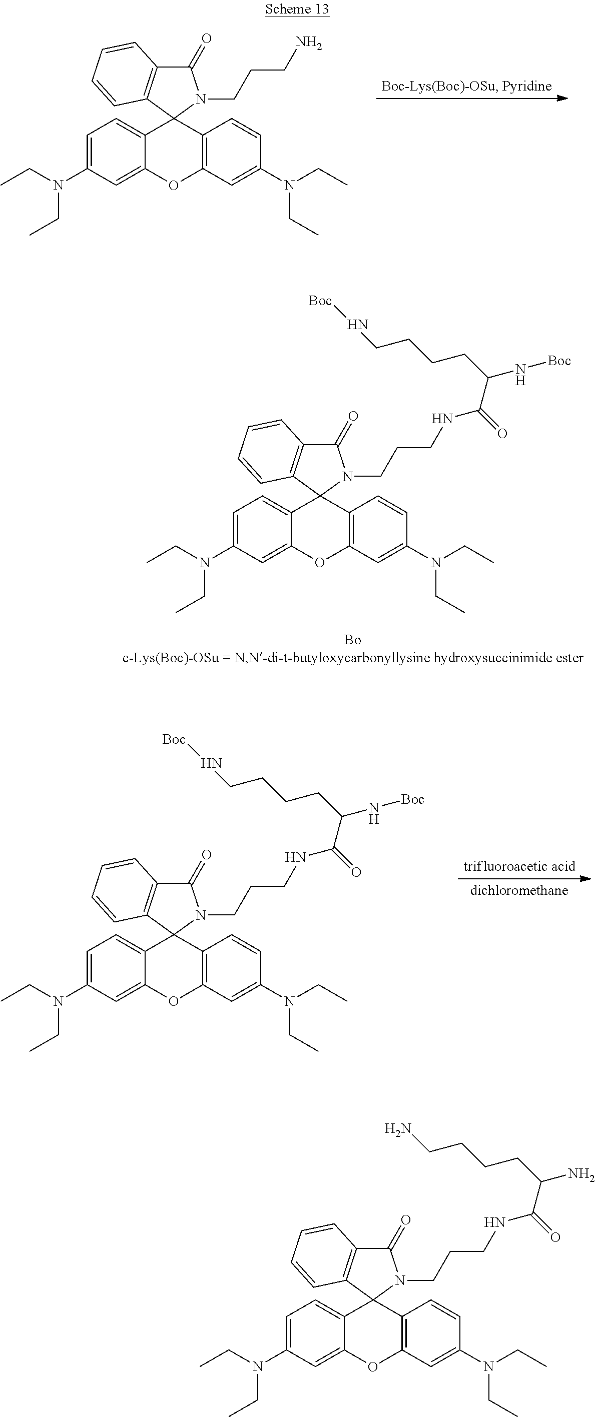

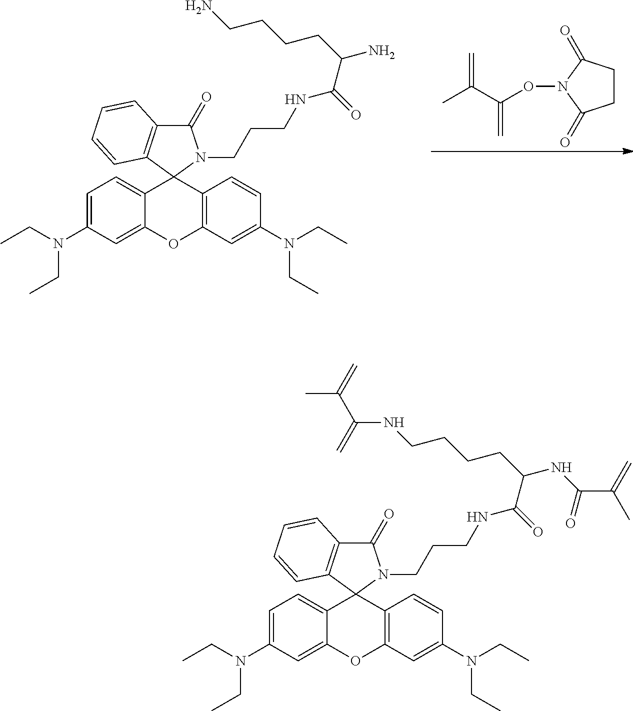

B. Benzospiropyran Derivatives

A dimethacryloyl ester benzospiropyran derivative is prepared, as described in Scheme 14:

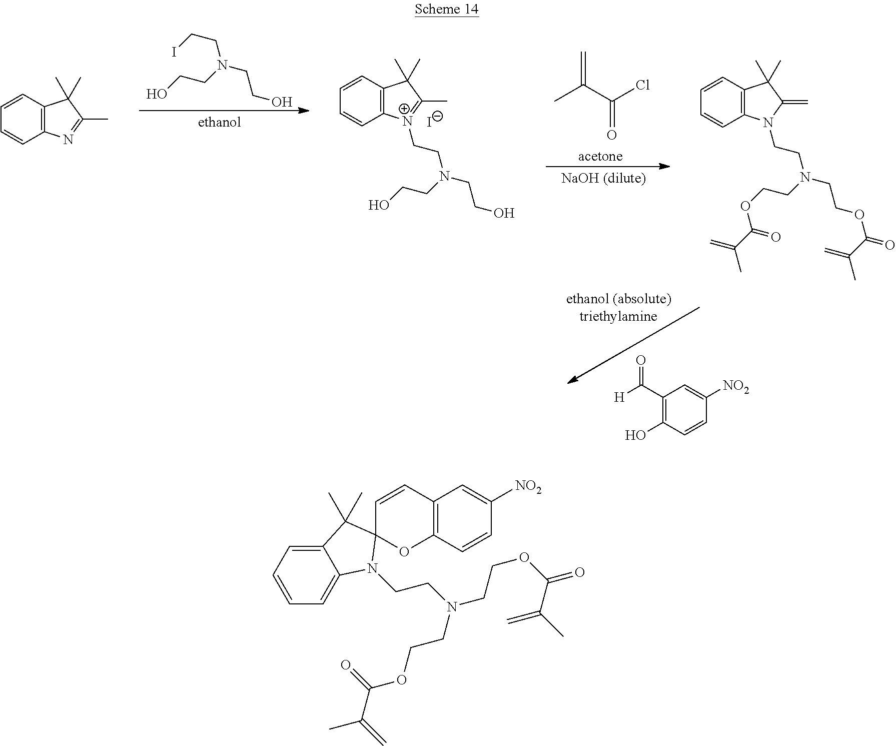

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-63. (canceled)

64. A molecularly imprinted polymer comprising a plurality of monomeric units, at least one of said monomeric units is a responsive monomeric unit being capable of undergoing a physico-chemical change in response to an external change, said responsive monomeric unit being incorporated within or attached to the molecularly imprinted polymer, the molecularly imprinted polymer being capable of selectively binding to a target molecule and releasing a bound target molecule in response to said external change.

65. The molecularly imprinted polymer of claim 64, wherein said physico-chemical change is selected from the group consisting of a change in electric charge, a change in polarity, a change in conformation, and a change in configuration.

66. The molecularly imprinted polymer of claim 64, wherein said external change is selected from the group consisting of a presence and/or change in concentration of a chemical, a change in pH, an exposure to light, an exposure to irradiation, a temperature change, an exposure to an electric current, and an exposure to an electromagnetic field.

67. The molecularly imprinted polymer of claim 64, wherein said physico-chemical change is reversible.

68. The molecularly imprinted polymer of claim 64, wherein said responsive monomeric unit comprises at least one polymerizable moiety and a responsive moiety, said responsive moiety being selected capable of undergoing a physico-chemical change in response to said external change, and said polymerizable moiety forming a polymeric backbone of the polymer by linking said monomeric units in said plurality of monomeric units to one another.

69. The molecularly imprinted polymer of claim 68, wherein said responsive moiety comprises a heteroalicyclic ring, said heteroalicyclic ring comprising a responsive bond linking a carbon atom and a heteroatom, wherein said responsive bond cleaves in response to said external change, such that said external change causes opening of said heteroalicyclic ring.

70. The molecularly imprinted polymer of claim 69, wherein said carbon atom which is linked by said responsive bond is further linked to an electron donating moiety.

71. The molecularly imprinted polymer of claim 70, wherein said responsive moiety has the general formula I: 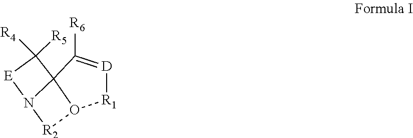
wherein:
the dashed lines denote that the oxygen atom is bound to either R₁ or R₂;
D is selected from the group consisting of N and CR₃;
E is an aromatic or heteroaromatic moiety, being substituted or non-substituted;
R₁ and R₂ are each independently selected from the group consisting of hydrogen, alkyl, alkenyl, alkynyl, cycloalkyl, heteroalicyclic, aryl, arylalkyl, and heteroaryl, or R₁ attaches to R₆ to form a 5- or 6-membered cycloalkyl, heteroalicyclic, aromatic or heteroaromatic ring; and
R₃-R₆ are each independently selected from the group consisting of hydrogen, alkyl, alkenyl, alkynyl, cycloalkyl, heteroalicyclic, aryl, heteroaryl, arylalkyl, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl, or R₃ attaches to R₆ to form a 5- or 6-membered cycloalkyl, heteroalicyclic, aromatic or heteroaromatic ring, with the proviso that neither R₄ nor R₅ is hydrogen.

72. The molecularly imprinted polymer of claim 70, wherein said responsive moiety has the general formula II: 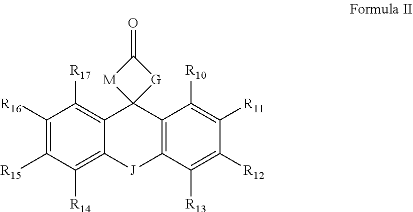
wherein:
G is selected from the group consisting of O, S and NR₁₉;
J is selected from the group consisting of O, S and NR₁₈;
M is an aromatic or heteroaromatic moiety, being substituted or non-substituted; and
R₁₀-R₁₇ are each independently selected from the group consisting of hydrogen, alkyl, alkenyl, alkynyl, cycloalkyl, heteroalicyclic, aryl, heteroaryl, arylalkyl, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl; and
R₁₈ and R₁₉ are each independently selected from the group consisting of hydrogen, alkyl, alkenyl, alkynyl, cycloalkyl, heteroalicyclic, aryl and heteroaryl.

73. The molecularly imprinted polymer of claim 68, wherein said responsive moiety has the general formula III: 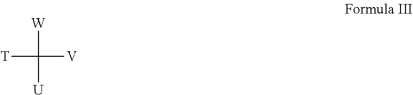
wherein:
T, U and V are each independently selected from the group consisting of substituted or non-substituted aromatic moiety and substituted or non-substituted heteroaromatic moiety; and
W is selected from the group consisting of hydroxy, thiohydroxy, halide, carboxy, and sulfonate.

74. A process for producing a molecularly imprinted polymer according to claim 64, the process comprising polymerizing a plurality of monomers in the presence of a template molecule, wherein at least one of said monomers is a responsive monomer being capable of undergoing a physico-chemical change in response to said external change, thereby producing said molecularly imprinted polymer, wherein said template molecule is similar or identical to said target molecule.

75. The process of claim 74, wherein said responsive monomer comprises at least one polymerizable moiety and a responsive moiety, said responsive moiety being selected capable of undergoing a physico-chemical change in response to said external change, and said polymerizable moiety being selected capable of linking to other monomers so as to form said polymer.

76. The process of claim 74, wherein said polymerizing is performed in a mixture of said plurality of monomers and said template molecule.

77. The process of claim 74, wherein said responsive monomer comprises a moiety derived from said target molecule and/or said template molecule, wherein after said polymerizing said moiety derived from said target molecule and/or said template molecule is released.

78. The process of claim 74, wherein said responsive moiety comprises a heteroalicyclic ring, said heteroalicyclic ring comprising a responsive bond linking a carbon atom and a heteroatom, wherein said responsive bond becomes cleaved in response to said external change, such that said external change causes opening of said heteroalicyclic ring.

79. The process of claim 74, wherein the responsive monomer is selected from the group consisting of:
A-L-B and 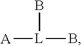
wherein:
A is said responsive moiety;
B is said polymerizable moiety; and
L is absent or is a linker moiety.

80. The process of claim 74, wherein said plurality of monomers comprise, in addition to said responsive monomer, a plurality of monomers comprising at least one polymerizable moiety.

81. A responsive monomer for preparing a molecularly imprinted polymer capable of selectively binding to a target molecule and releasing a bound target molecule in response to an external change, the monomer comprising at least one polymerizable moiety, and a responsive moiety comprising a heteroalicyclic ring, said heteroalicyclic ring comprising a responsive bond linking a carbon atom and a heteroatom, wherein said responsive bond becomes cleaved in response to said external change, such that said external change causes opening of said heteroalicyclic ring.

82. The responsive monomer of claim 81, wherein said carbon atom which is linked by said responsive bond is further linked to an electron donating moiety.

83. The responsive monomer of claim 82, wherein said responsive moiety has the general formula I: 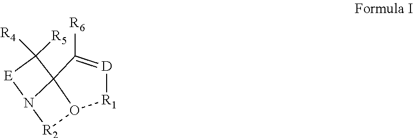
wherein:
the dashed lines denote that the oxygen atom is bound to either R₁ or R₂;
D is selected from the group consisting of N and CR₃;
E is an aromatic or heteroaromatic moiety, being substituted or non-substituted;
R₁ and R₂ are each independently selected from the group consisting of hydrogen, alkyl, alkenyl, alkynyl, cycloalkyl, heteroalicyclic, aryl, arylalkyl, and heteroaryl, or R₁ attaches to R₆ to form a 5- or 6-membered cycloalkyl, heteroalicyclic, aromatic or heteroaromatic ring; and
R₃-R₆ are each independently selected from the group consisting of hydrogen, alkyl, alkenyl, alkynyl, cycloalkyl, heteroalicyclic, aryl, heteroaryl, arylalkyl, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl, or R₃ attaches to R₆ to form a 5- or 6-membered cycloalkyl, heteroalicyclic, aromatic or heteroaromatic ring, with the proviso that neither R₄ nor R₅ is hydrogen.

84. The responsive monomer of claim 82, wherein said responsive moiety has the general formula II: 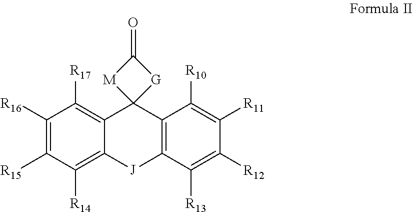
wherein:
G is selected from the group consisting of O, S and NR₁₉;
J is selected from the group consisting of O, S and NR₁₈;
M is an aromatic or heteroaromatic moiety, being substituted or non-substituted; and
R₁₀-R₁₇ are each independently selected from the group consisting of hydrogen, alkyl, alkenyl, alkynyl, cycloalkyl, heteroalicyclic, aryl, heteroaryl, arylalkyl, hydroxy, alkoxy, aryloxy, thiohydroxy, thioalkoxy, thioaryloxy, halide, amine, amide, carbonyl, carboxy, thiocarboxy, carbonyl, thiocarbonyl, sulfonate, sulfate, urea, disulfide, epoxide (oxirane), sulfonyl, sulfinyl, sulfonamide, nitro, nitrile, isonitrile, thiirane, aziridine, nitroso, hydrazine, carbamyl and thiocarbamyl; and
R₁₈ and R₁₉ are each independently selected from the group consisting of hydrogen, alkyl, alkenyl, alkynyl, cycloalkyl, heteroalicyclic, aryl and heteroaryl.

85. A responsive monomer for preparing a molecularly imprinted polymer capable of selectively binding to a target molecule and releasing a bound target molecule in response to an external change, the monomer comprising at least one polymerizable moiety, and a responsive moiety having the general formula III: 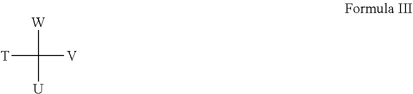
wherein:
T, U and V are each independently selected from the group consisting of substituted or non-substituted aromatic moiety and substituted or non-substituted heteroaromatic moiety; and
W is selected from the group consisting of hydroxy, thiohydroxy, halide, carboxy, and sulfonate.

86. The responsive monomer of claim 81, wherein the responsive monomer is selected from the group consisting of:
A-L-B and 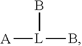
wherein:
A is said responsive moiety;
B is said polymerizable moiety; and
L is absent or is a linker moiety.

87. The responsive monomer of claim 81, wherein said responsive monomer comprises a moiety derived from said target molecule, wherein after said polymerizing said moiety derived from said target molecule is released.

88. The responsive monomer of claim 87, wherein said responsive monomer is selected from the group consisting of: 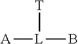
and T-L-A-L-B
wherein:
A is said responsive moiety;
B is said polymerizable moiety;
L is absent or is a linker moiety; and
T is a moiety derived from said target molecule.

89. The responsive monomer of claim 85, wherein said responsive monomer comprises a moiety derived from said target molecule, wherein after said polymerizing said moiety derived from said target molecule is released.

90. The responsive monomer of claim 89, wherein said responsive monomer is selected from the group consisting of: 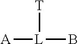
and T-L-A-L-B
wherein:
A is said responsive moiety;
B is said polymerizable moiety;
L is absent or is a linker moiety; and
T is a moiety derived from said target molecule.

91. A molecularly imprinted polymer produced according to the process of claim 74.

92. The molecularly imprinted polymer of claim 64, wherein said plurality of monomeric units comprises, in addition to said at least one responsive monomeric unit, a plurality of monomeric units which comprise at least one polymerizable moiety for forming a polymeric backbone of said polymer by linking said monomeric units in said plurality of monomeric units to one another.

93. The molecularly imprinted polymer of claim 64, being for reversibly binding said target molecule.

94. A method of selectively and reversibly binding a target molecule, the method comprising contacting the molecularly imprinted polymer of claim 64 with said target molecule.

95. The molecularly imprinted polymer of claim 94, wherein said target molecule is a biological or non-biological marker.
